(19) United States
(12) Patent Application Publication
(10) Pub. No.: US 2020/0197442 A1
(43) Pub. Date: Jun. 25, 2020

(54) PHARMACEUTICAL COMPOSITION FOR PREVENTION OR TREATMENT OF ALZHEIMER'S DISEASE, COMPRISING STEM CELL SECRETING SRAGE

(71) Applicant: NSAGE CORP., Incheon (KR)

(72) Inventors: Bonghee LEE, Jeju-si (KR); Kyung Hee BYUN, Incheon (KR); Delger Bayarsaikhan, Ulaanbaatar (MN); Jaeseok LEE, Incheon (KR); Myeongjoo SON, Incheon (KR); Hosseinisalkadeh Seyedghasem, Teheran (IR)

(21) Appl. No.: 16/608,813

(22) PCT Filed: Apr. 26, 2018
(86) PCT No.: PCT/KR2018/004874

§ 371 Date: Oct. 25, 2019

(30) Foreign Application Priority Data
Apr. 26, 2017 (KR) 10-2017-0053922

(51) Int. Cl. A61K 35/28; C12N 5/074; C12N 5/0775; A61P 25/28

(52) U.S. Cl. CPC A61K 35/28; A61P 25/28; C12N 5/0663; C12N 5/0696

(57) ABSTRACT

Provided are soluble RAGE-secreting stem cells and a medicinal use thereof in preventing and/or treating Alzheimer's disease.

### TECHNICAL FIELD

Provided are sRAGE-secreting stem cell and a use thereof for preventing and/treating Alzheimer's disease.

### BACKGROUND ART

Alzheimer's disease (AD) is one of the most common neurodegenerative diseases, mainly exhibiting dementia symptoms although different symptoms predominantly arise in the late phase thereof. AD patients are neuropathologically characterized by the existence of intracellular neurofibrillary tangles and extracellular amyloid plaques composed of amyloid beta (Aβ) in the brain thereof. AP denotes peptides of about 39-43 amino acids. The peptides derive from the amyloid precursor protein, which is cleaved to yield Aβ. Aβ₁₋₄₀ is a major soluble AP species in biological fluids while Aβ₁₋₄₂ (minor soluble Aβ species) is the more fibrillogenic than Aβ₁₋₄₀ and plays an important role in the pathogenesis of AD.

### DETAILED DESCRIPTION OF THE INVENTION

### Technical Problem

Proposed in this disclosure are a sRAGE-secreting stem cell created by gene editing technology for suppressing RAGE expression in an Aβ₁₋₄₂-injected AD rat model, a RAGE expression inhibiting effect thereof in an Aβ₁₋₄₂-injected AD rat model, and a use thereof in treating Alzheimer's disease.

An embodiment provides a stem cell secreting a soluble receptor for advanced glycation end products (sRAGE). The stem cell may be a stem cell harboring a sRAGE-encoding gene thereat, for example, a stem cell to which a sRAGE-encoding gene is transduced by gene editing.

Another embodiment provides a method for preparing a sRAGE-secreting stem cell, the method comprising a step of introducing a sRAGE-encoding gene into a stem cell. The preparation method may further comprise a step of culturing the stem cell having the sRAGE-encoding gene introduced thereinto to express the sRAGE (inside the stem cell) and/or releasing the sRAGE (outside the stem cell), after the introducing step.

Another embodiment provides a pharmaceutical composition comprising the sRAGE-secreting stem cell for prevention and/or treatment of Alzheimer's disease. Another embodiment provides a use of the sRAGE-secreting stem cell in preventing or treating Alzheimer's disease. Another embodiment provides a method for prevention or treatment of Alzheimer's disease, the method comprising a step of administering the sRAGE-secreting stem cell to a patient in need thereof. The method for prevention and/or treatment of Alzheimer's disease may further comprise a step of identifying a patient in need of prevention and/or treatment of Alzheimer's disease, before the administering step.

Another embodiment provides a pharmaceutical composition comprising the sRAGE-secreting stem cell for suppressing expression of a RAGE ligand and/or an inflammatory protein in a patient with Alzheimer's disease. Another embodiment provides a use of sRAGE-secreting stem cells in suppressing expression of a RAGE ligand and/or an inflammatory protein in a patient with Alzheimer's disease. Another embodiment provides a method for suppressing expression of a RAGE ligand and/or an inflammatory protein in a patient with Alzheimer's disease, the method comprising a step of administering the sRAGE-secreting stem cell to the patient.

Another embodiment provides a pharmaceutical composition comprising the sRAGE-secreting stem cell for inhibiting RAGE-mediated neuronal death and/or inflammation in a patient with Alzheimer's disease. Another embodiment provides a use of the sRAGE-secreting stem cell in inhibiting RAGE-mediated neuronal death and/or inflammation in a patient with Alzheimer's disease. Another embodiment provides a method for inhibiting RAGE-mediated neuronal death and/or inflammation in a patient with Alzheimer's disease, the method comprising a step of administering the sRAGE-secreting stem cell to the patient.

### Technical Solution

Alzheimer's disease (AD) is among the most common neurodegenerative diseases and is postulated to be caused by amyloid beta (Aβ) deposits, which lead to neuronal loss and synaptic dysfunction. AP promotes the synthesis and secretion of Receptor for Advanced Glycation End products (RAGE) ligands upon activation of microglial cells and was found to cause neuronal death in AD mouse models. Meanwhile, soluble RAGE (sRAGE) reduces inflammation and decreases microglial activation and AP deposition to deter neuronal death. However, sRAGE is too short in half-life to be used for therapeutic purposes. Provided in this disclosure are sRAGE-secreting stem cells (sRAGE-secreting MSCs) that repress Aβ deposition and reduce the synthesis and secretion of a RAGE ligand in an Aβ₁₋₄₂-induced AD model. In addition, sRAGE-secreting MSCs were found to deter RAGE/RAGE ligand binding in an Aβ₁₋₄₂-induced AD model, showing an enhanced in-vivo survival rate and an elevated protective effect. This result implies that sRAGE-secreting stem cells could be an effective means for protecting neurons against Alzheimer's disease, with the protective effect attributed to the deterrence of RAGE-mediated cell death or inflammation

An embodiment provides a soluble Receptor for Advanced Glycation End products (sRAGE)-secreting stem cell. The stem cell may be a stem cell harboring a sRAGE-encoding gene therein, for example, a stem cell to which a sRAGE-encoding gene is introduced by gene editing (e.g., DNA scissors, etc.). The sRAGE-encoding gene may be inserted into the safe harbor gene site in the genome of the stem cell. To this end, the gene editing technology may be designed to target and cleave the safe harbor gene.

Another embodiment provides a method for preparing a sRAGE-secreting stem cell, the method comprising a step of introducing a sRAGE-encoding gene into a stem cell. The preparation method may further comprise a step of culturing the stem cell having the sRAGE-encoding gene introduced thereinto to express the sRAGE (inside the stem cell) and/or releasing the sRAGE (outside the stem cell), after the introducing step. The step of introducing a sRAGE-encoding gene may be conducted by a gene editing technology (e.g., gene scissors, etc.). As stated above, the gene editing technology may be designed to target and cleave the safe harbor gene.

Another embodiment provides a pharmaceutical composition comprising the sRAGE-secreting stem cell or a culture of the stem cell for prevention and/or treatment of Alzheimer's disease. Another embodiment provides a use of the sRAGE-secreting stem cell or a culture of the stem cell in preventing or treating Alzheimer's disease. Another embodiment provides a method for prevention or treatment of Alzheimer's disease, the method comprising a step of administering the sRAGE-secreting stem cell or a culture of the stem cell to a patient in need thereof. The method for prevention and/or treatment of Alzheimer's disease may further comprise a step of identifying a patient in need of prevention and/or treatment of Alzheimer's disease, before the administering step.

The sRAGE-secreting stem cell (or a culture thereof) or the pharmaceutical composition comprising the same is characterized by having inhibitory activity against the expression of amyloid precursor protein (APP) and/or beta-site APP cleaving enzyme 1 (BACE1), the expression of a RAGE ligand and/or an inflammatory protein, and/or RAGE-mediated neuronal death and/or inflammation in a patient with Alzheimer's disease.

Another embodiment provides a pharmaceutical composition comprising the sRAGE-secreting stem cell for downregulating expression of amyloid precursor protein (APP) and/or beta-site APP cleaving enzyme 1 (BACE1) in a patient with Alzheimer's disease. Another embodiment provides a use of the sRAGE-secreting stem cell in downregulating expression of amyloid precursor protein (APP) and/or beta-site APP cleaving enzyme 1 (BACE1) in a patient with Alzheimer's disease. Another embodiment provides a method for downregulating expression of amyloid precursor protein (APP) and/or beta-site APP cleaving enzyme 1 (BACE1) in a patient Alzheimer's disease, the method comprising a step of administering the sRAGE-secreting stem cell to the patient.

Another embodiment provides a pharmaceutical composition comprising the sRAGE-secreting stem cell for downregulating the expression of a RAGE ligand and/or an inflammatory protein in a patient with Alzheimer's disease. Another embodiment provides a use of the sRAGE-secreting stem cell in downregulating the expression of a RAGE ligand and/or an inflammatory protein in a patient with Alzheimer's disease. Another embodiment provides a method for downregulating expression of a RAGE ligand and/or an inflammatory protein in a patient with Alzheimer's disease, the method comprising a step of administering the sRAGE-secreting stem cell to the patient. The RAGE ligand may be at least one selected from AGE (Advanced Glycation End products), HMGB1 (High mobility group box 1), and S100β, but is not limited thereto.

Another embodiment provides a pharmaceutical composition comprising the sRAGE-secreting stem cell for inhibiting RAGE-mediated neuronal death and/or inflammation in a patient with Alzheimer's disease. Another embodiment provides a use of the sRAGE-secreting stem cell in inhibiting RAGE-mediated neuronal death and/or inflammation in a patient with Alzheimer's disease. Another embodiment provides a method for inhibiting RAGE-mediated neuronal death and/or inflammation in a patient with Alzheimer's disease, the method comprising a step of administering the sRAGE-secreting stem cell to the patient.

Hereinafter, a detailed description will be given of the present disclosure:

The patient may be selected from mammals including primates such as humans, apes, and the like and rodents such as rats, mice, and the like, which suffer from Alzheimer's disease, cells (brain cells) or tissues (brain tissues) isolated from the mammals, or cultures thereof. By way of example, selection may be made of a human suffering from Alzheimer's disease, brain cells or tissues isolated therefrom, or a culture of the cells or tissues.

The sRAGE-secreting stem cell provided as an effective ingredient in the disclosure or a pharmaceutical composition comprising the same may be administered via various routes including oral and parenteral routes. For example, the cells or composition may be administered in any convenient way, such as injection, transfusion, implantation, or transplantation into a lesion site (e.g., brain) of a patient with Alzheimer's disease, or via vessel routes (vein or artery), without any limitation thereto.

The pharmaceutical compositions provided herein may be formulated according to conventional methods into oral dosage forms such as powders, granules, tablets, capsules, suspensions, emulsions, syrups, aerosols, or parenteral dosage forms such as suspensions, emulsions, lyophilized agent, external preparations, suppositories, sterile injectable solutions, implant preparations and the like.

The amount of the composition of the present disclosure may vary depending on the age, sex, and weight of the subject to be treated, and above all, the condition of the subject to be treated, the specific category or type of cancer to be treated, the route of administration, the nature of the therapeutic agent used, and the sensitivity to specific therapeutic agents, and may be prescribed in consideration thereof. For example, the stem cells may be administered to an Alzheimer's disease patient at a dose of 1×10³−1×10⁹ cells, e.g., 1×10⁴−1×10⁸ cells or 1×10⁵−1×10⁷ cells per kg of body weight, but is not limited thereto.

The sRAGE may be derived from mammals including primates humans, apes, and the like and rodents such as rats, mice, and the like. In one embodiment, the sRAGE may be at least one selected from the group consisting of the human sRAGE proteins (GenBank Accession Nos: NP_001127.1 (gene: NM_001136.4) [Q15109-1], NP_001193858.1 (gene: NM_001206929.1) [Q15109-6], NP_001193861.1 (gene: NM_001206932.1) [Q15109-7], NP_001193863.1 (gene: NM_001206934.1) [Q15109-4], NP_001193865.1 (gene: NM_001206936.1) [Q15109-9], NP_001193869.1 (gene: NM_001206940.1) [Q15109-3], NP_001193883.1 (gene: NM_001206954.1) [Q15109-8], NP_001193895.1 (gene: NM_001206966.1) [Q15109-3], and NP_751947.1 (gene: NM_172197.2) [Q15109-2]), but is not limited thereto.

As used herein, the term “stem cell” is intended to encompass all embryonic stem cells, adult stem cells, induced pluripotent stem cells (iPS cells), and progenitor cells. For example, the stem cell may be at least one selected from the group consisting of embryonic stem cells, adult stem cells, induced pluripotent stem cells, and progenitor cells.

Embryonic stem cells are stem cells derived from an embryo and able to differentiate into cells of any tissue.

Induced pluripotent stem cells (iPS cells), also called dedifferentiated stem cells, are embryonic-like pluripotent cells that are generated by injecting a cell differentiation related gene into differentiated somatic cells to reprogram the somatic cells back to a pre-differentiation cell state.

Progenitor cells have a tendency to differentiate into a specific type of cells, but are already more specific than stem cells and are pushed to differentiate into their target cells. Unlike stem cells, progenitor cells undergo limited divisions. The progenitor cells may be derived from mesenchymal stem cells, but are not limited thereto. In the disclosure, progenitor cells fall within the scope of stem cells and unless otherwise stated, “stem cells” are construed to include progenitor cells.

Adult stem cells, which are stem cells derived from the umbilical cord, umbilical cord blood or adult bone marrow, blood, nerves, etc., refer to primitive cells immediately before differentiation into cells of concrete organs. The adult stem cells are at least one selected from the group consisting of hematopoietic stem cells, mesenchymal stem cells, neural stem cells, and the like.

Adult stem cells are difficult to proliferate and are prone to differentiation. Instead, adult stem cells can be used not only to reproduce various organs required by actual medicine, but also to differentiate according to the characteristics of individual organs after transplantation thereto. Hence, adult stem cells can be advantageously applied to the treatment of incurable diseases.

In one embodiment, the adult stem cells may be mesenchymal stem cells (MSC). The term “mesenchymal stem cells”, also called mesenchymal stromal cells (MSC), means multipotent stromal cells that can differentiate into various types of cells, such as osteoblasts, chondrocytes, myocytes, adipocytes, and the like. Mesenchymal stem cells may be selected from pluripotent cells derived from non-marrow tissues such as placenta, umbilical cord, umbilical cord blood, adipose tissues, adult muscles, corneal stroma, and dental pulp from deciduous teeth.

The stem cell may be a human-derived stem cell.

The sRAGE-secreting stem cell may be at least one selected from the group consisting of human-derived sRAGE-secreting mesenchymal stem cells (hereinafter referred to as “human RAGE-secreting MSC”) and human-derived sRAGE-secreting induced pluripotent stem cells (hereinafter referred to as “human sRAGE-secreting iPSC”).

The sRAGE-secreting stem cell may be stem cells, e.g., mesenchymal stem cells or induced pluripotent stem cells, having the sRAGE-encoding gene inserted to the genome thereof.

In one embodiment, the sRAGE-encoding gene may be inserted into a safe harbor gene site in the genome of the stem cells. A safe harbor gene site is a genomic location where DNA may be damaged (cleaved, and/or deletion, substitution, or insertion of nucleotide(s)) without disrupting cell injury may include, but is not limited to, AAVS1 (adeno-associated virus integration site; e.g., AAVS1 in human chromosome 19(19q13)).

Insertion (introduction) of the sRAGE-encoding gene into a stem cell genome may be achieved using any genetic manipulation technique that is typically used to introduce a gene into a genome in animal cells. In one embodiment, the genetic manipulation technique may employ target-specific nuclease. The target-specific nuclease may target such a safe harbor gene site as is described above.

As used herein, the term “target-specific nuclease”, which is also called programmable nuclease, is intended to encompass all types of nucleases (e.g., endonucleases) that recognize and cleave specific sites on target genomic DNA. The target-specific nuclease may be an enzyme isolated from a microbe or a non-naturally occurring enzyme obtained in a recombinant or synthetic manner. The target-specific nuclease may further include an element that is typically used for intracellular delivery in eukaryotic cells (e.g., nuclear localization signal; NLS), but is not limited thereto. The target specific nuclease may be used in the form of a purified protein, a DNA encoding the same, or a recombinant vector carrying the DNA.

For example, the target-specific nuclease may be at least one selected from the group consisting of:

transcription activator-like effector nuclease (TALEN) in which a transcription activator-like (TAL) effector DNA-binding domain, derived from a gene responsible for plant infection, for recognizing a specific target sequence is fused to a DNA cleavage domain;

zinc-finger nuclease (ZEN);

meganuclease;

RNA-guided engineered nuclease (RGEN), which is derived from the microbial immune system CRISPR, such as Cas proteins (e.g., Cas9, etc.), Cpf1, and the like; and

Ago homolog (DNA-guided endonuclease), but is not limited thereto.

The target-specific nuclease recognizes specific base sequences in the genome of animal and plant cells (i.e., eukaryotic cells), including human cells, to cause double strand breaks (DSBs). The double strand breaks create a blunt end or a cohesive end by cleaving the double strands of DNA. DSBs are efficiently repaired by homologous recombination or non-homologous end-joining (NHEJ) mechanisms within the cell, which allows researchers to introduce desired mutations into on-target sites during this process.

The meganuclease may be included within, but is not limited to, a scope of naturally occurring meganucleases. The naturally occurring meganucleases recognize 15-40 base pair-long sites to be cleaved and are commonly classified into families: LAGUDADG family, GIY-YIG family, His-Cyst box family, and HNH family. Exemplary meganucleases include I-SceI, I-CeuI, PI-PspI, PI-SceI, I-SceIV, I-CsmI, I-PanI, I-SceII, I-PpoI, I-SceIII, I-CreI, I-TevI, I-TevII, and

DNA-binding domains from naturally occurring meganucleases, primarily from the LAGLIDADG family, have been used to promote site-specific genome modification in plants, yeasts, Drosophila, mammalian cells, and mice, but this approach has been limited to the modification of either homologous genes that conserve the meganuclease recognition sequence (Monet et al. (1999), Biochem. Biophysics. Res. Common. 255: 88-93) or pre-engineered genomes into which a recognition sequence has been introduced. Accordingly, attempts have been made to engineer meganucleases to exhibit novel binding specificity at medically or biotechnologically relevant sites. In addition, naturally occurring or engineered DNA-binding domains from meganucleases have been operably linked to a cleavage domain from a heterologous nuclease (e.g., Fold).

The ZFN comprises a zinc finger protein engineered to bind to a target site in a gene of interest and cleavage domain or a cleavage half-domain. The ZFN may be an artificial restriction enzyme comprising a zinc-finger DNA binding domain and a DNA cleavage domain. Here, the zinc-finger DNA binding domain may be engineered to bind to a sequence of interest. For example, reference may be made to Beerli et al. (2002) Nature Biotechnol. 20:135-141; Pabo et al. (2001) Ann. Rev. Biochem. 70:313-340; Isalan et al, (2001) Nature Biotechnol. 19: 656-660; Segal et al. (2001) Curr. Opin. Biotechnol. 12:632-637; and Choo et al. (2000) Curr. Opin. Struct. Biol. 10:411-416. Compared to a naturally occurring zinc finger protein, an engineered zinc finger binding domain can have a novel binding specificity. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence.

Selection of target sites, and design and construction of fusion proteins (and polynucleotides encoding the same) are known to those skilled in the art and described in detail in U.S. Patent Nos. 2005/0064474 A and 2006/0188987 A, incorporated by reference in their entireties herein. In addition, as disclosed in these and other references, zinc finger domains and/or multi-fingered zinc finger proteins may be linked together using any suitable linker sequences, including, for example, linkers of 5 or more amino acids in length. Reference may be made to U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Nucleases such as ZFNs also comprise a nuclease active site (cleavage domain, cleavage half-domain) As noted above, the cleavage domain may be heterologous to the DNA-binding domain, for example, such as a zinc finger DNA-binding domain and a cleavage domain from a different nuclease. Heterologous cleavage domains can be obtained from any endonuclease or exonuclease. Exemplary endonucleases from which a cleavage domain can be derived include, but are not limited to, restriction endonucleases and meganucleases.

Similarly, a cleavage half-domain can be derived from any nuclease or portion thereof, as set forth above, which requires dimerization for cleavage activity. In general, two fusion proteins are required for cleavage if the fusion proteins comprise cleavage half-domains Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in an embodiment, the near edges of the target sites are separated by 5-8 nucleotides or by 14-18 nucleotides.

However, any integral number of nucleotides or nucleotide pairs can intervene between two target sites (e.g., from 2 to 50 nucleotide pairs or more). Generally, the site of cleavage lies between the target sites.

Restriction endonucleases (restriction enzymes) are present in many species and are capable of binding to DNA (at a recognition site) in a sequence-specific manner and cleaving DNA at or near the site of binding. Certain restriction enzymes (e.g., Type RS) cleave DNA at sites removed from the recognition site and have separable binding and cleavage domains For example, the Type RS enzyme Fok I catalyzes double-stranded cleavage of DNA, at 9 nucleotides from its recognition site on one strand and 13 nucleotides from its recognition site on the other. Thus, in one embodiment, fusion proteins comprise the cleavage domain (or cleavage half-domain) from at least one Type RS restriction enzyme and one or more zinc finger binding domains (which may or may not be engineered).

As used herein, the term “TALEN” refers to a nuclease capable of recognizing and cleaving a target region of DNA. TALEN is a fusion protein comprising a TALE domain and a nucleotide cleavage domain. In the present disclosure, the terms “TAL effector nuclease” and “TALEN” are interchangeably used. TAL effectors are known as proteins that are secreted by Xanthomonas bacteria via their type lit secretion system when they infect a variety of plant species. The protein may be bound to a promoter sequence in a host plant to activate the expression of a plant gene that aids bacterial infection. The protein recognizes plant DNA sequences through a central repetitive domain consisting of various numbers of 34 or fewer amino acid repeats. Accordingly, TALE is considered to be a novel platform for tools in genome engineering. However, in order to construct a functional TALEN with genomic-editing activity, a few key parameters that have remained unknown thus far should be defined as follows: i) the minimum DNA-binding domain of TALE, the length of the spacer between the two half-sites constituting one target region, and the linker or fusion junction that links the Fold nuclease domain to dTALE.

The TALE domain of the present disclosure refers to a protein domain that binds nucleotides in a sequence-specific manner via one or more TALE-repeat modules. The TALE domain includes, but is not limited to, at least one TALE-repeat module, and more specifically, 1 to 30 TALE-repeat modules. In the present disclosure, the terms “TAL effector domain” and “TALE domain” are interchangeable. The TALE domain may include half of the TALE-repeat module. As concerns the TALEN, reference may be made to Patent Publication No. WO/2012/093833 or U.S. Patent No. 2013-0217131 A of which the entire contents are incorporated by reference in their entireties herein.

In one embodiment, insertion (or introduction) of the sRAGE-encoding gene into a stem cell genome may be achieved using a target-specific nuclease (RGEN derived from CRISPR). The target-specific nuclease may comprise:

(1) an RNA-guided nuclease (or a DNA coding therefore or a recombinant vector carrying the coding DNA), and

(2) a guide RNA capable of hybridizing with (or having a complementary nucleotide sequence to) a target site (e.g., a region of 15 to 30, 17 to 23, or 18 to 22 consecutive nucleotides in a safe harbor gene such as AAVS1) in a target gene (e.g., a safe harbor site such as AAVS1), or a DNA coding therefor (or a recombinant vector carrying the coding DNA).

For example, the target-specific nuclease may be at least one selected from all nucleases that can recognize specific sequences of target genes and have nucleotide cleavage activity to incur indel (insertion and/or deletion) in the target genes.

In one embodiment, the target-specific nuclease may be at least one selected from the group consisting of nucleases (e.g., endonucleases) included in the type II and/or type V CRISPR system, such as Cas proteins (e.g., Cas9 protein (CRISPR (Clustered regularly interspaced short palindromic repeats) associated protein 9)), Cpfl protein (CRISPR from Prevotella and Francisella 1), etc. In this regard, the target-specific nuclease further comprises a target DNA-specific guide RNA for guiding to a target site on a genomic DNA. The guide RNA may be an RNA transcribed in vitro, for example, RNA transcribed from double-stranded oligonucleotides or a plasmid template, but is not limited thereto. The target-specific nuclease may act in a ribonucleoprotein (RNP) form in which the nuclease is associated with guide RNA to form a ribonucleic acid-protein complex (RNA-Guided Engineered Nuclease), in vitro or after transfer to a body (cell).

The Cas protein, which is a main protein component in the CRISPR/Cas system, accounts for activated endonuclease or nickase activity.

The Cas protein or gene information may be obtained from a well-known database such as GenBank at the NCBI (National Center for Biotechnology Information). By way of example, the Cas protein may be at least one selected from the group consisting of:

a Cas protein derived from Streptococcus sp., e.g., Streptococcus pyogenes, for example, Cas9 protein (i.e., SwissProt Accession number Q99ZW2 (NP_269215.1));

a Cas protein derived from Campylobacter sp., e.g., Campylobacter jejuni, for example, Cas9 protein;

a Cas protein derived from Streptococcus sp., e.g., Streptococcus thermophiles or Streptococcus aureus, for example, Cas9 protein;

a Cas protein derived from Neisseria meningitidis, for example, Cas9 protein;

a Cas protein derived from Pasteurella sp., e.g., Pasteurella multocida, for example, Cas9 protein; and

a Cas protein derived from Francisella sp., e.g., Francisella novicida, for example, Cas9 protein, but is not limited thereto.

The Cpf1 protein, which is an endonuclease in a new CRISPR system distinguished from the CRISPR/Cas system, is small in size relative to Cas9, requires no tracrRNA, and can act with the guidance of single guide RNA. In addition, the Cpfl protein recognizes a thymine-rich PAM (protospacer-adjacent motif) sequence and cleaves DNA double strands to form a cohesive end (cohesive double-strand break).

By way of example, the Cpfl protein may be derived from Candidatus spp., Lachnospira spp., Butyrivibrio spp., Peregrinibacteria, Acidominococcus spp., Porphyromonas spp., Prevotella spp., Francisella spp., Candidatus Methanoplasma, or Eubacterium spp., e.g., from Parcubacteria bacterium (GWC2011_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum, Candidatus Paceibacter, Eubacterium eligens, etc., but is not limited thereto.

The target-specific nuclease may be isolated from microbes or may be an artificial or non-naturally occurring enzyme as obtained by recombination or synthesis. For use, the target-specific nuclease may be in the form of an mRNA pre-described or a protein pre-produced in vitro or may be included in a recombinant vector so as to be expressed in target cells or in vivo. In an embodiment, the target-specific nuclease (e.g., Cas9, Cpf1, etc.) may be a recombinant protein made with a recombinant DNA (rDNA). The term “recombinant DNA” means a DNA molecule formed by artificial methods of genetic recombination, such as molecular cloning, to bring together homologous or heterologous genetic materials from multiple sources. For use in producing a target-specific nuclease by expression in a suitable organism (in vivo or in vitro), recombinant DNA may have a nucleotide sequence that is reconstituted with optimal codons for expression in the organism which are selected from codons coding for a protein to be produced.

The target-specific nuclease used herein may be a mutant target-specific nuclease in an altered form. The mutant target-specific nuclease may refer to a target-specific nuclease mutated to lack the endonuclease activity of cleaving double strand DNA and may be, for example, at least one selected from among mutant target-specific nucleases mutated to lack endonuclease activity but to retain nickase activity and mutant target-specific nucleases mutated to lack both endonuclease and nickase activities. As such, the mutation of the target-specific nuclease (e.g., amino acid substitution, etc.) may occur at least in the catalytically active domain of the nuclease (for example, RuvC catalyst domain for Cas9). In an embodiment, when the target-specific nuclease is a Streptococcus pyogenes-derived Cas9 protein (SwissProt Accession number Q99ZW2(NP_269215.1); SEQ ID NO: 4), the mutation may be amino acid substitution at one or more positions selected from the group consisting of a catalytic aspartate residue (e.g., aspartic acid at position 10 (D10) for SEQ ID NO: 4, etc.), glutamic acid at position 762 (E762), histidine at position 840 (H840), asparagine at position 854 (N854), asparagine at position 863 (N863), and aspartic acid at position 986 (D986) on the sequence of SEQ ID NO: 4. A different amino acid to be substituted for the amino acid residues may be alanine, but is not limited thereto.

In another embodiment, the mutant target-specific nuclease may be a mutant that recognizes a PAM sequence different from that recognized by wild-type Cas9 protein. For example, the mutant target-specific nuclease may be a mutant in which at least one, for example, all of the three amino acid residues of aspartic acid at position 1135 (D1135), arginine at position 1335 (R1335), and threonine at position 1337 (T1337) of the Streptococcus pyogenes-derived Cas9 protein are substituted with different amino acids to recognize NGA (N is any residue selected from among A, T, G, and C) different from the PAM sequence (NGG) of wild-type Cas9.

In one embodiment, the mutant target-specific nuclease may have the amino acid sequence (SEQ ID NO: 4) of Streptococcus pyogenes-derived Cas9 protein on which amino acid substitution has been made for:
(1) D10, H840, or D10 +H840;
(2) D1135, R1335, T1337, or D1135 +R1335 +T1337; or
(3) both of (1) and (2) residues.

As used herein, the term “a different amino acid” means an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all variants thereof, exclusive of the amino acid retained at the original mutation positions in wild-type proteins. In one embodiment, “a different amino acid” may be alanine, valine, glutamine, or arginine

As used herein, the term “guide RNA” refers to an RNA that includes a targeting sequence hybridizable with a specific base sequence (target sequence) of a target site in a target gene and functions to associate with a nuclease, such as Cas proteins, Cpf1, etc., and to guide the nuclease to a target gene (or target site) in vitro or in vivo (or in cells).

The guide RNA may be suitably selected depending on kinds of the nuclease to be complexed therewith and/or origin microorganisms thereof.

For example, the guide RNA may be at least one selected from the group consisting of: CRISPR RNA (crRNA) including a region (targeting sequence) hybridizable with a target sequence;

trans-activating crRNA (tracrRNA) including a region interacting with a nuclease such as Cas protein, Cpf1, etc.; and

single guide RNA (sgRNA) in which main regions of crRNA and tracrRNA (e.g., a crRNA region including a targeting sequence and a tracrRNA region interacting with nuclease) are fused to each other.

In detail, the guide RNA may be a dual RNA including CRISPR RNA (crRNA) and trans-activating crRNA (tracrRNA) or a single guide RNA (sgRNA) including main regions of crRNA and tracrRNA.

The sgRNA may include a region (named “spacer region”, “target DNA recognition sequence”, “base pairing region”, etc.) having a complementary sequence (targeting sequence) to a target sequence in a target gene (target site), and a hairpin structure for binding to a Cas protein. In greater detail, the sgRNA may include a region having a complementary sequence (targeting sequence) to a target sequence in a target gene, a hairpin structure for binding to a Cas protein, and a terminator sequence. These moieties may exist sequentially in the direction from 5′ to 3′, but without limitations thereto. So long as it includes main regions of crRNA and tracrRNA and a complementary sequence to a target DNA, any guide RNA can be used in the present disclosure.

For editing a target gene, for example, the Cas9 protein requires two guide RNAs, that is, a CRISPR RNA (crRNA) having a nucleotide sequence hybridizable with a target site in the target gene and a trans-activating crRNA (tracrRNA) interacting with the Cas9 protein. In this context, the crRNA and the tracrRNA may be coupled to each other to form a crRNA:tracrRNA duplex or connected to each other via a linker so that the RNAs can be used in the form of a single guide RNA (sgRNA). In one embodiment, when a Streptococcus pyogenes-derived Cas9 protein is used, the sgRNA may form a hairpin structure (stem-loop structure) in which the entirety or a part of the crRNA having a hybridizable nucleotide sequence is connected to the entirety or a part of the tracrRNA including an interacting region with the Cas9 protein via a linker (responsible for the loop structure).

The guide RNA, specially, crRNA or sgRNA, includes a targeting sequence complementary to a target sequence in a target gene and may contain one or more, for example, 1-10, 1-5, or 1-3 additional nucleotides at an upstream region of crRNA or sgRNA, particularly at the 5′ end of sgRNA or the 5′ end of crRNA of dual RNA. The additional nucleotide(s) may be guanine(s) (G), but are not limited thereto.

In another embodiment, when the nuclease is Cpfl, the guide RNA may include crRNA and may be appropriately selected, depending on kinds of the Cpfl protein to be complexed therewith and/or origin microorganisms thereof.

Concrete sequences of the guide RNA may be appropriately selected depending on kinds of the nuclease (Cas9 or Cpf1) (i.e., origin microorganisms thereof) and are an optional matter which could easily be understood by a person skilled in the art.

In an embodiment, when a Streptococcus pyogenes-derived Cas9 protein is used as a target-specific nuclease, crRNA may be represented by the following General Formula 1:

(General Formula 1)
5′-(N_{cas9})₁-(GUUUUAGAGCUA)-(X_{cas9})m-3′

wherein:

N_{cas9} is a targeting sequence, that is, a region determined according to a sequence at a target site in a target gene (i.e., a sequence hybridizable with a sequence of a target site), 1 represents a number of nucleotides included in the targeting sequence and may be an integer of 15 to 30, 17 to 23 or 18 to 22, for example, 20;

the region including 12 consecutive nucleotides (GUUUUAGAGCUA; SEQ ID NO: 1) adjacent to the 3′-end of the targeting sequence is essential for crRNA;

X_{cas9} is a region including m nucleotides present at the 3′-terminal site of crRNA (that is, present adjacent to the 3′-end of the essential region); and

m may be an integer of 8 to 12, for example, 11 wherein the m nucleotides may be the same or different and are independently selected from the group consisting of A, U, C, and G.

In an embodiment, the X_{cas9} may include, but is not limited to, UGCUGUUUUG (SEQ ID NO: 2).

In addition, the tracrRNA may be represented by the following General Formula 2:

(General Formula 2)
5′-(Y_{cas9})ₚ-(UAGCAAGUUAAAAUAAGGCUAGUCCGUUAUCAACUUGAA
AAAGUGGCACCGAGUCGGUGC)-3′

wherein,

the region represented by 60 nucleotides (UAGCAAGUUAAAAUAAGGCUAGUCCGUUAUCAACUUGAAAAAGUGGCACC GAGUCGGUGC) (SEQ ID NO: 3) is essential for tracrRNA,

Y_{cas9} is a region including p nucleotides present adjacent to the 3′-end of the essential region, and

p is an integer of 6 to 20, for example, 8 to 19 wherein the p nucleotides may be the same or different and are independently selected from the group consisting of A, U, C, and G.

Further, sgRNA may form a hairpin structure (stem-loop structure) in which a crRNA moiety including the targeting sequence and the essential region of the crRNA and a tracrRNA moiety including the essential region (60 nucleotides) of the tracrRNA are connected to each other via an oligonucleotide linker (responsible for the loop structure). In greater detail, the sgRNA may have a hairpin structure in which a crRNA moiety including the targeting sequence and an essential region of crRNA is coupled with the tracrRNA moiety including the essential region of tracrRNA to form a double-strand RNA molecule with connection between the 3′ end of the crRNA moiety and the 5′ end of the tracrRNA moiety via an oligonucleotide linker.

In one embodiment, the sgRNA may be represented by the following General Formula 3:

(General Formula 3)
5′-(N_{cas9})₁-(GUUUUAGAGCUA)-(oligonuc1eotide linker)-
(UAGCAAGUUAAAAUAAGGCUAGUCCGUUAUCAACUUGAAAAAGUGGCACC
GAGUCGGUGC)-3′

wherein (N_{cas9})₁ is a targeting sequence defined as in General Formula 1.

The oligonucleotide linker included in the sgRNA may be 3-5 nucleotides long, for example 4 nucleotides long in which the nucleotides may be the same or different and are independently selected from the group consisting of A, U, C, and G.

The crRNA or sgRNA may further contain 1 to 3 guanines (G) at the 5′ end thereof (that is, the 5′ end of the targeting sequence of crRNA).

The tracrRNA or sgRNA may further comprise a terminator inclusive of 5 to 7 uracil (U) residues at the 3′ end of the essential region (60 nt long) of tracrRNA.

The target sequence for the guide RNA may be about 17 to about 23 or about 18 to about 22, for example, 20 consecutive nucleotides adjacent to the 5′ end of PAM (Protospacer Adjacent Motif (for S. pyogenes Cas9, 5′-NGG-3′ (N is A, T, G, or C)) on a target DNA.

As used herein, the term “the targeting sequence” of guide RNA hybridizable with the target sequence for the guide RNA refers to a nucleotide sequence having a sequence complementarity of 50% or higher, 60% or higher, 70% or higher, 80% or higher, 90% or higher, 95% or higher, 99% or higher, or 100% to a nucleotide sequence of a complementary strand to a DNA strand on which the target sequence exists (i.e., a DNA strand having a PAM sequence (5′-NGG-3′ (N is A, T, G, or C))) and thus can complimentarily couple with a nucleotide sequence of the complementary strand.

In another embodiment, when the target-specific nuclease is a Cpf1 system, the guide RNA (crRNA) may be represented by the following General Formula 4:

(General Formula 4)
5′-n1-n2-A-U-n3-U-C-U-A-C-U-n4-n5-n6-n7-G-U-A-G-A-
U-(Ncpf1)q-3′

wherein,

n1 is null or represents U, A, or G,

n2 represents A or G,

n3 represents U, A, or C,

n4 is null or represents G, C, or A,

n5 represents A, U, C, or G, or is null,

n6 represents U, G, or C,

n7 represents U or G,

Ncpf1 is a targeting sequence including a nucleotide sequence hybridizable with a target site on a target gene and is determined depending on the target sequence of the target gene, and

q represents a number of nucleotides included therein and may be an integer of 15 to 30.

The target sequence (hybridizing with crRNA) of the target gene is a 15 to 30 (e.g., consecutive) nucleotide-long sequence adjacent to the 3′ end of PAM (5′-TTN-3′ or 5′-TTTN-3′; N is any nucleotide selected from A, T, G, and C.

In General Formula 4, the 5 nucleotides from the 6^{th} to the 10^{th} position from the 5′ end (5′ terminal stem region) and the 5 nucleotides from the 15^{th} (16^{th} when n4 is not null) to the 19^{th} (20^{th} when n4 is not null) from the 5′ end are complementary to each other in the antiparallel manner to form a duplex (stem structure), with the concomitant formation of a loop structure composed of 3 to 5 nucleotides between the 5′ terminal stem region and the 3′ terminal stem region.

For the Cpfl protein, the crRNA (e.g., represented by General Formula 4) may further comprise 1 to 3 guanine residues (G) at the 5′ end.

In crRNA sequences for Cpfl proteins available from microbes of Cpfl origin, 5′ terminal sequences (exclusive of targeting sequence regions) are illustratively listed in Table 1.

TABLE 1

| | 5′ Terminal Sequence |
|---|---|
| | (5′-3′) of guide |
| Microbe of Cpf1 origin | RNA (crRNA) |
| Parcubacteria bacterium GWC2011_GWC2_44_17 (PbCpfl) | AAAUUUCUACU-UUUGUAGAU |
| Peregrinibacteria bacterium GW2011_GWA_33_10 (PeCpf1) | GGAUUUCUACU-UUUGUAGAU |
| Acidaminococcus sp. BVBLG (AsCpf1) | UAAUUUCUACU-CUUGUAGAU |
| Porphyromonas macacae (PmCpf1) | UAAUUUCUACU-AUUGUAGAU |
| Lachnospiraceae bacterium ND2006 (LbCpi1) | GAAUUUCUACU-AUUGUAGAU |
| Porphyromonas crevioricanis (PcCpf1) | UAAUUUCUACU-AUUGUAGAU |
| Prevotella disiens (PdCpf1) | UAAUUUCUACU-UCGGUAGAU |
| Moraxella bovoculi 237 (MbCpf1) | AAAUUUCUACUGUUUGUAGAU |
| Leptospira inadai (LiCpf1) | GAAUUUCUACU-UUUGUAGAU |
| Lachnospiraceae bacterium MA2020 (Lb2Cpf1) | GAAUUUCUACU-AUUGUAGAU |
| Francisella novicida U112 (FnCpf1) | UAAUUUCUACU-GUUGUAGAU |
| Candidatus Methanoplasma termitum (CMtCpf1) | GAAUCUCUACUCUUUGUAGAU |
| Eubacterium eligens (EeCpf1) | UAAUUUCUACU--UUGUAGAU |

(-: denotes the absence of any nucleotide)

As used herein, the term “nucleotide sequence” hybridizable with a gene target site refers to a nucleotide sequence having a sequence complementarity of 50% or higher, 60% or higher, 70% or higher, 80% or higher, 90% or higher, 95% or higher, 99% or higher, or 100% to a nucleotide sequence (target sequence) of the gene target site (hereinafter used in the same meaning unless otherwise stated. The sequence homology can be using a typical sequence comparison mean (e.g., BLAST)).

RAGE and its ligands are important targets for Alzheimer's disease-related inflammation Soluble RAGE (sRAGE) is an extracellular domain of RAGE and can interrupt the extracellular binding between RAGE and the ligands thereof.

However, the interruption of RAGE by sRAGE in vivo is limited: (1) sRAGE is short in in-vivo half-life. For example, when introduced into the brain of a patient with Alzheimer's disease (AD), sRAGE protein is rapidly degraded; (2) in addition, sRAGE inhibits RAGE-dependent inflammation in AD, but some inflammation mediator of AD cannot be interrupted by sRAGE; and (3) attempts have been made to treat Alzheimer's disease by transplantation of mesenchymal stem cells (MSCs). Secreting various cytotropic factors, MSCs promote cell growth, reduce cell death, and increase autophagy. In addition, MSC transplantation regulates microglia-related neuritis to reduce Aβ deposition. However, the transplanted MSCs also express on cell surfaces RAGE which is, in turn, bound to a ligand thereof to cause a RAGE cascade.

In order to solve the problem of the short half-life sRAGE protein, sRAGE-secreting MSCs (sRAGE-MSCs) or sRAGE-secreting iPSCs (sRAGE-iPSCs) were established and sRAGE-secreting MSCs were representatively tested for the efficacy in the brains of Aβ₁₋₄₂-injected rats according to one embodiment of the present disclosure. As a result, brains of Aβ₁₋₄₂-injected rats following treatment with sRAGE-MSC were observed to increase the viability of the injected MSCs and reduce Aβ deposition, inflammation, and neuronal death, compared to those following treatment with MSCs (FIGS. 2a, 3a, 3b, and 6).

With the treatment with sRAGE-MSC, sRAGE was secreted at a concentration of 0.011 pg per cell, which is in a lower level than that of the treated sRAGE protein (FIG. 1g). In addition, treatment with sRAGE-MSCs is more effective than treatment with sRAGE because sRAGE-MSC continuously secretes sRAGE over many passages (FIGS. 8a and 8b). Furthermore, the genetic modification of human MSC with sRAGE did not alter stemness characteristics (FIG. 1h). The secreted sRAGE downregulates RAGE expression, thereby protecting MSC from cell death (FIGS. 1d-1g). A longer survival duration of the stem cells was detected in the brains of Aβ₁₋₄₂-injected rats treated with sRAGE-MSC than with MSC (FIGS. 1b and 1c). These results indicate that sRAGE interrupts RAGE-ligand binding on MSCs in the brain of Aβ₁₋₄₂-injected rats, suggesting the plausibility of acting as a niche controller in an Aβ₁₋₄₂-induced environment.

AGE (advanced glycation endproducts; RAGE ligand) increases microglial synthesis in a vicious feedback cycle, which further aggravates the AD conditions through increased AP production by upregulating the BACE1 level. In an embodiment, sRAGE-MSC was found to reduce BACE1 (beta-secretase 1) levels and AP deposition in brains of Aβ₁₋₄₂-injected rats as measured by immunofluorescence and immunoblot analysis (FIGS. 3a to 3e). Exposure to Aβ₁₋₄₂ induces microglial activation which leads to the expression of RAGE ligands. The number of activated microglial cells were decreased in brains of the Aβ₁₋₄₂-injected rats after sRAGE-MSC treatment (FIGS. 4a and 4b). In addition to the activation of microglial cells, the expression level of RAGE ligands was further decreased after treatment with sRAGE-MSC than sRAGE or MSC (FIG. 4e). In order to determine the source of the RAGE ligands, a human microglical cell line (HMO6) was activated with Aβ₁₋₄₂-exposed SH-SYSY neuron medium (CM). A sRAGE protein, an MSC medium, and a sRAGE medium were fed to the CM-treated HMO6 cells. The sRAGE secreted from sRAGE-MSC not only downregulates the expression of RAGE ligands in the supernatant, cell lysates, and animal tissues (FIGS. 4c and 4d) but also mitigates interaction between RAGE and the ligand thereof (FIGS. 5b to 5d).

AGE, HMGB 1 (high mobility group box 1), and S100β (dominant RAGE ligands) are involved in neuronal diseases and the interaction thereof with the receptor induces RAGE cascade which is associated with inflammation and neuronal apoptosis. The sRAGE secreted by sRAGE-MSC suppressed RAGE-ligand binding and markedly reduced the levels of RAGE-related inflammation and apoptosis-related molecules such as pSAPK/JNK (phosphorylated stress-activated protein kinase/c-Jun N-terminal kinase) in the brains of Aβ₁₋₄₂-injected rats. In addition, sRAGE secreted by sRAGE-MSC markedly reduced caspase 3, caspase 8, caspase 9, and NF (nuclear factors), too, in the brains of Aβ₁₋₄₂-injected rats, compared to sRAGE protein or MSC (FIGS. 4c, 5, and 6). Post-intracellular stress, calcium influx-induced activation of protein kinases, such as SAPK/JNK, may cause apoptotic cell death. In an embodiment of the present disclosure, examination was made of the effect of Aβ₁₋₄₂ on cell death, SAPK/JNK, and caspases in the brains of Aβ₁₋₄₂-injected rats. As a result, sRAGE-MSC was found to have protective activity against the neuronal apoptosis promoted by the inactivation of the SAPK/JNK-Caspase pathway (FIGS. 5b and 6). Finally, it was found that sRAGE-MSCs are superior to sRAGE or MSCs in terms of protective effect on neuronal apoptosis in the brains of Aβ₁₋₄₂-injected rats (FIG. 6).

### Advantageous Effect

As provided in the present disclosure, sRAGE-secreting MSC may be used as an effective ingredient to effectively diminish AO deposition and microglial activation, compared to sRAGE or MSC, in the brain of Alzheimer's disease animal models. In addition, sRAGE-secreting MSC can remarkably decline a RAGE ligand level and interaction between RAGE and RAGE ligands, compared to sRAGE or MSC, in activated microglial cells. Furthermore, sRAGE-MSCs can continuously secrete sRAGE, resulting in a neuronal protective effect in the brain of an Alzheimer's disease model (e.g., Aβ₁₋₄₂-injected rat). Accordingly, sRAGE-secreting MSC can fmd advantageous applications in the prevention and/or treatment of Alzheimer's disease.

### BRIEF DESCRIPTION OF THE DRAWINGS

FIG. 1 illustratively shows properties of sRAGE-secreting MSCs,

FIG. 1a is a cleavage map of an expression vector available for CRISPR-mediated production of sRAGE-secreting MSCs (sRAGE-MSCs),

FIG. 1b depicts a genomic DNA (gDNA) of the sRAGE secreted from sRAGE-MSCs as measured by junction PCR using a sRAGE-specific nucleotide sequence,

FIG. 1c is a graph showing a level of sRAGE in a conditioned medium (CM) of 1.5×10⁶ sRAGE-MSCs, as measured by ELISA,

FIGS. 1d and 1e show immunoblotting results accounting for sRAGE-conjugated Flag expression levels in sRAGE-MSC supernatant (d) and cell lysate (e).

FIG. 1f provides dual staining confocal microscopic images showing expressions of the sRAGE conjugated Flag (Flag, red) and stem cell markers (Endoglin, green) (Scale bar=100 nm, magnification=200×),

FIG. 1g is a graph showing Flag intensities in representative confocal microscopy images of FIG. If (**, <0.01 versus MSC, ***, <0.001 versus MSC),

FIG. 1h provides cytograms showing expression levels of positive stem cell markers (CD44, CD73) and negative markers (CD34) of sRAGE-MSCs.

FIG. 2 shows an increase in the viability of sRAGE-MSCs through the interruption of RAGE-induced cell death (as confirmed by immunofluorescence and qRT-PCR analysis four weeks after final transplantation of MSCs and sRAGE-MSCs into the brains of Aβ₁₋₄₂-injected rats),

FIG. 2a depicts immunofluorescence images accounting for the distribution of CD44-positive cells (red) as analyzed by immunofluorescence,

FIG. 2b is a graph showing the number of CD44-positive cells,

FIGS. 2c to 2e are graphs of expression levels of the human specific stem cell markers (CD44 gene (2c), CD90 gene (2d), and CD117 gene (2e)) as analyzed by qRT-PCR ((*, <0.05 versus MSC-treated Aβ₁₋₄₂ injected rat brains), in which expression levels of respective gene markers are expressed as fold values relative to the expression level of GADPH gene in the rat,

FIG. 2f depicts fluorescence images accounting for RAGE expression (green) in MSCs and sRAGE-MSCs after treatment with 1 μM Aβ₁₋₄₂ or PBS for 96 hrs., as analyzed by immunofluorescence,

FIG. 2g is a graph in which the intensity of immunofluorescence obtained in FIG. 2f is quantitated,

FIG. 2h depicts fluorescence images accounting for apoptosis (red) of MSCs and sRAGE-MSCs, as analyzed by TUNEL,

FIG. 2i is a graph showing the ratios (%) of apoptotic cells to total cells (in FIG. 2, scale bar=100 μm, *, <0.05, ***, <0.001, versus MSC group. DAPI stained nuclei are blue colored).

FIG. 3 shows expression levels of amyloid precursor protein (APP) and beta-secretase 1 (BACE1) in Aβ₁₋₄₂-injected rat brains after treatment sRAGE-MSC,

FIG. 3a depicts fluorescence images accounting for APP expression (green), observed under a confocal microscope (Scale bar=100 μm),

FIG. 3b is a graph showing mean values after quantifying APP expression (green) fluorescence intensities observed in FIG. 3a for the cells, with no statistically significant differences between Aβ₁₋₄₂-injected rat brains treated with MSCs and sRAGE-MSCs,

FIG. 3c depicts fluorescence images accounting for BACE1 expression (green) observed under a confocal microscope (Scale bar=100 μm),

FIG. 3d is a graph showing mean values after quantifying APP expression (green) fluorescence intensities observed in FIG. 3c for the cells, with no statistically significant differences between Aβ₁₋₄₂-injected rat brains treated with MSCs and sRAGE protein,

FIG. 3e depicts APP and BACE1 protein levels in the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment, as measured by immunoblotting analysis (in FIG. 3, §§§, <0.001, versus naive controls, ***, <0.001, versus Aβ₁₋₄₂ injected rat brains, ##<0.01, ###, <0.001, versus sRAGE-MSC treated Aβ₁₋₄₂ injected rat brains).

FIG. 4 shows that sRAGE-MSC treatment induces microglial activation and the downregulation of inflammation-related proteins including RAGE ligands in vivo and in vitro,

FIG. 4a depicts confocal microscopic images accounting for the distribution of activated microglial cells (Iba1 ; green) in the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment (nuclei was DAPI stained and appeared blue) (Scale bar=100 μm),

FIG. 4b is a graph showing the ratio of Iba1-positive cells to total cells expressed (<0.001, versus naive controls, ***, <0.001, versus Aβ₁₋₄₂ injected rat brains, ###, <0.001, versus sRAGE-MSC treated Aβ₁₋₄₂ injected rat brains), with no significant differences between the Aβ₁₋₄₂-injected rat brains treated with sRAGE protein and MSCs,

FIG. 4c shows the expression levels of inflammatory proteins including IL-Iβ and NF-κB in the brains of Aβ₁₋₄₂-injected rats, as measured by immunoblotting analysis, with iNOS and Argl used for M1 and M2 markers, respectively,

FIG. 4d is a graph showing levels of the RAGE ligands AGE, HMGB1 and S100β in HM06 cell lysates after treatment with CM, sRAGE protein, MSC medium (MSC med), or sRAGE-MSC medium (sRAGE-MSC med) for 24 hours, respectively, as analyzed by ELISA,

FIG. 4e is a graph showing levels of AGE, HMGB1, and S100β in brain tissues of Aβ₁₋₄₂-injected rats (in FIGS. 4d and 4e, §, <0.05, versus nave controls, *, <0.05, versus Aβ₁₋₄₂ injected rat brains, #<0.05, versus sRAGE-MSC treated Aβ₁₋₄₂ injected rat brains).

FIG. 5 shows that sRAGE-MSC treatment inhibits [symbol] RAGE-related apoptosis pathway and downregulates RAGE expression in Aβ₁₋₄₂-injected rat brains,

FIG. 5a depicts confocal microscopic images accounting for RAGE expression (green) in the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment (nuclei was DAPI stained and appeared blue) (Scale bar=100 μm),

FIG. 5b shows SAPK/JNK, pSAPK/JNK, caspase 3, caspase 8, and caspase 9 in the brains of rat having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment, as measured by immunoblotting analysis.

FIG. 6 shows that sRAGE-MSC treatment inhibits RAGE-mediated neuronal apoptosis inβ₁₋₄₂-injected rat brains,

FIG. 6a depicts confocal micrographic images of the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment, with TUNEL-positive cells appearing red (Scale bar=100 μm),

FIG. 6b is a graph showing counts of TNNEL-positive cells as measured by image J software,

FIG. 6c shows images of cresyl violet staining accounting for neuronal populations (Scale bar=200 μm),

FIG. 6d is a graph showing counts of stained cells as measured by image J software (§, <0.05, versus naive controls, *, <0.05, versus Aβ₁₋₄₂ injected rat brains, #<0.05, versus sRAGE-MSC treated Aβ₁₋₄₂ injected rat brains).

FIGS. 7a to 7f show sequence alignments of the sRAGE produced from sRAGE-MSCs.

FIG. 8 shows the expression of sRAGE-conjugated Flag in MSCs, backbone vector pZD/MSCs, and sRAGE-MSCs,

FIG. 8a shows confocal microscopic images of sRAGE-conjugated Flag (red), nuclei (DAPI, blue), and MSC-specific markers (Endoglin, green) in MSCs, pZD-MSCs, and subcultured sRAGE-MSCs (S1, S2, and S3),

FIG. 8b is a graph in which Flag expression is quantified from the representative result of FIG. 8a (pZD/MSC; pZDonor-AAVS1 backbone vector transfected MSC, S1; first cell subculture, S2; second cell subculture, S3; third cell subculture Scale bar=100 μm, Data are means±Standard Deviation, ***, significantly different (P<0.001), NS; Not Significant).

FIG. 9 shows characteristics of sRAGE-secreting iPSCs,

FIG. 9a depicts an image of electrophoresis accounting for a PCR product of sRAGE-encoding gene transfected into iPSCs with the aid of pZDonor-AAVS1 vector, and

FIG. 9b depicts the expression and secretion levels of sRAGE, as measured by western blotting and ELISA.

### MODE FOR CARRYING OUT THE INVENTION

Hereinafter, the present disclosure will be described in more detail with reference to Examples, which are merely illustrative and are not intended to limit the scope of the present disclosure. It is apparent to those skilled in the art that the Examples described below may be modified without departing from the essential gist of the disclosure.

### REFERENCE EXAMPLE

1. Culture of MSCs, HMO6, and SH-SY5Y Cells

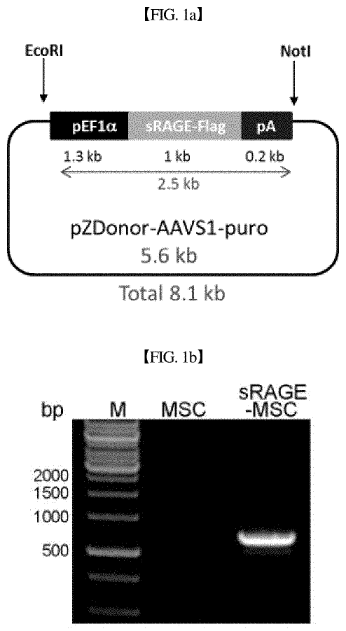

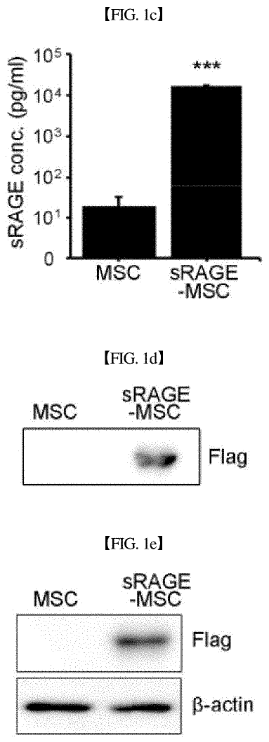

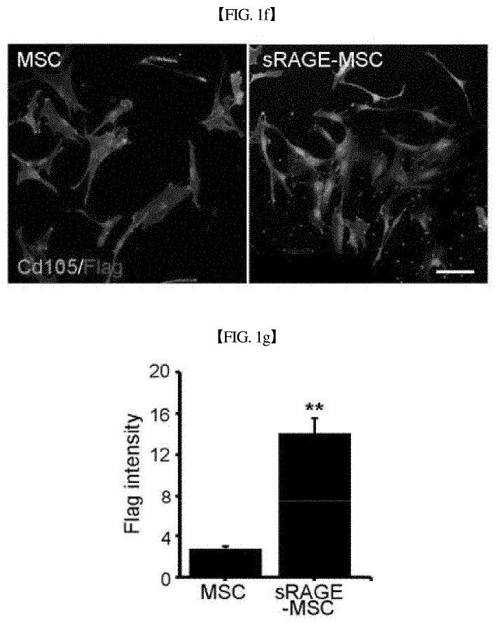

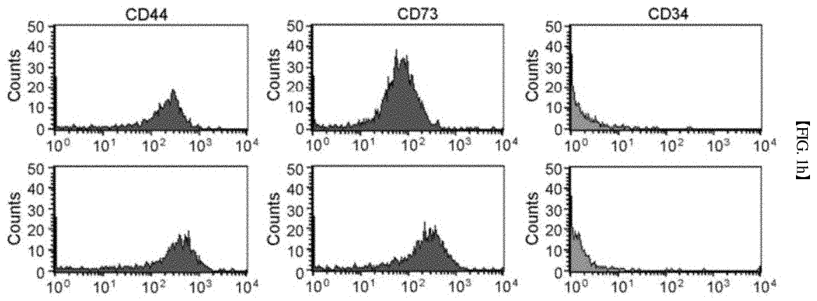

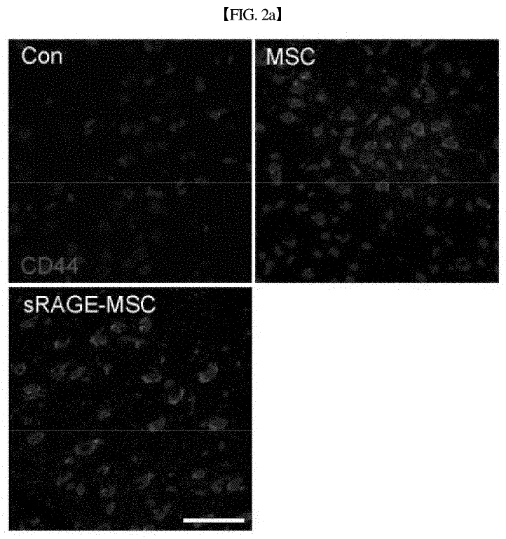

Human umbilical cord-mesenchymal stem cells (MSCs) were purchased from CEFObio (Seoul, Korea). sRAGE-secreting mesenchymal stem cells (sRAGE-MSCs) were prepared by introducing a donor vector carrying sRAGE (cat. RD172116100, Biovendor; SEQ ID NO: 6) (see FIG. 1a) into MSCs (CEFObio) (see Reference Example 2). For use in evaluating a paracrine effect (in vitro), the MSCs and the sRAGE-MSCs were cultured in an MSC medium (MSC med; DMEM, Gibco® Life Technologies Corp.) and a sRAGE-MSC medium (sRAGE-MSC med; used to culture sRAGE-secreting MSCs, DMEM, Gibco® Life Technologies Corp.), respectively, for two days. Both of the media were supplemented with proteinase inhibitor and phosphatase inhibitor (TAKARA, Tokyo, Japan) to prevent proteolysis. The MSC medium and the sRAGE-MSC medium were loaded to respective centrifugal filter units (Millipore, Merck Millipore, Germany) and centrifuged for 50 min at 3,220×g and 4° C. The concentrated media thus obtained were stored at 80° C. until use.

SH-SY5Y cells (human neuroblastoma cell line; ATCC CRL-2266) and HMO6 cells (microglial cell line) were subjected to neural cell test. SH-SY5Y cells were cultured in a minimum essential medium (Hyclone, South Logan, Utah) while HMO6 cells were cultured in Dulbecco's modified Eagle's medium (Hyclone). Both of the media contained 10% heat-inactivated fetal bovine serum (Hyclone) and 1% penicillin streptomycin (Hyclone). SH-SYSY cells (at 70% confluence) were cultured for 96 hours in a fresh medium containing 1 NM amyloid beta (Aβ₁₋₄₂; Sigma-Aldrich, St. Louis, Mo.). The SH-SY5Y conditioned medium (CM) thus obtained was collected and concentrated for MSC med and sRAGE-MSC med as described above. In order to investigate the synthesis and secretion of RAGE ligand from activated microglial cells, the HMO6 cells were treated with sRAGE protein, or concentrated MSC med or sRAGE-MSC med for 24 hours and then with CM for 24 hours. All the cells used in the following Examples were maintained at 37° C. in a 5% CO₂ incubator.

2. Establishment of sRAGE MSCs by Using CRISPR/Cas9

In order to construct sRAGE-secreting MSCs (Srage-MSCs), mRNA CRISPR/Cas9 targeting the safe harbor site of AAVS1 (adeno-associated virus integration site 1) (ToolGen, Inc; Cas9: derived from Streptococcus pyogenes (SEQ ID NO: 4), and AAVS1 target site of sgRNA: 5′-gtcaccaatcctgtccctag-3′ (SEQ ID NO: 7)) were transfected into AAVS1.

The sgRNA has the following nucleotide:

5′-(target target)-(GUUUUAGAGCUA; SEQ ID NO: 1)-
(nucleotide linker)-UAGCAAGUUAAAAUAAGGCUAGUCCG
(UUAUCAACUUGAAAAAGUGGCACCGAGUCGGUGC; SEQ ID NO:
3)-3′

(the target sequence is the sequence modified from the AAVS1 target site sequence by converting “T” to “U” and the nucleotide sequence has the sequence of GAAA).

In this regard, 10 μl of the sRAGE sequence (used in the form of the donor vector of FIG. 1a) and transfect substrates were used for nucleofection in the following condition; 1050 volts, pulse width 30, pulse number 2, NEON Microporator (Thermo Fisher Scientific, Waltham, Mass.). After being seeded onto a 60 mm-culture dish (BD Biosciences, San Jose, Calif.), 10⁶ cells were stabilized at 37° C. in a 5% CO2 incubator for 7 days before injection. The medium was freshly changed daily.

3. Specimen Preparation

3.1. Frozen Block Tissue Slide

A brain specimen was obtained. To this end, a rat was anesthetized and subject to transcardial perfusion with 200 mL of saline solution at 18° C. and then with 200 mL of 0.1 M phosphate buffered saline (PBS) containing 4% paraformaldehyde. The extracted brain was immersed at 4° C. for 4 hours in a fixative solution and then transferred to ice-chilled 0.1M PBS containing 20% sucrose (Sigma-Aldrich). The brain prepared as such was stored at −20° C. until use by sectioning into 10 or 30 μm-long tubes with a cryotome.

3.2. Protein Separation

In order to determine protein expression levels in vivo and in vitro, the prepared brain or cells were lysed using an EzRIPA lysis kit (ATTO, Tokyo). Subsequently, the entorhinal cortices (ENT) were homogenized, followed by centrifugation at 13,000×g for 20 min at 4° C. The supernatant was transferred to a new tube and analyzed for protein quantification using Bicinchoninic acid assay kit (Thermo Fisher Scientific).

3.3. RNA Isolation

Total RNA in rat cerebral fat was isolated using Trizol reagent (Thermo Fisher Scientific) according to the manufacturer's instruction. In brief, ENT was homogenized in 1 mL of Trizol reagent mixed with 0.2 mL of chloroform (Amresco, Solon, OH) and centrifuged at 12,000×g for 15 min at 4° C. The supernatant was transferred into a new tube, mixed with 0.5 mL of 100% isopropanol, and centrifuged at 12,000×g for 10 min. The RNA pellet thus obtained was washed with 70% ethanol and spun down at 7,500×g for 5 min. After being dried, the pellet was dissolved in 30 μl of diethylpyrocarbonate (DEPC) water and quantified using Nanodrop 2000 (Thermo Fisher Scientific).

4. Junction PCR for Examining sRAGE Secretion from MSCs

In order to examine sRAGE expression in MSCs, genomic DNAs (gDNAs) of MSC and sRAGE-secreting MSC (sRAGE-MSC) was extracted using GeneJET genomic DNA purification kit (Thermo Fisher Scientific). Thereafter, concentrations of the gDNAs were measured using Nanodrop 2000. Equivalent amounts of the gDNAs were amplified by PCR in the following condition: 15 cycles of denaturation (30 sec at 90° C.) and annealing (90 sec at 68° C.) and 20 cycles of denaturation (30 sec at 95° C.), annealing (30 sec at 58° C.) and synthesis (90 sec at 72° C.), followed by a primer extension (5 mins at 72° C.). Primer sequences used for the PCR are summarized in Table 2.

TABLE 2
Junction PCR and Primer Sequences used
for qRT-PCR

| Gene | | Sequence |
|---|---|---|
| AAVS1 | Forward | 5′-CGG AAC TCT GCC CTC TAA CG-3′ |
| PURO | Reverse | 5′-TGA GGA AGA GTT CTT GCA GCT-3′ |
| CD44 | Forward | 5′-CCT TTG ATG GAC CAA TTA CCA T-3′ |
| | Reverse | 5′-GGG TAG ATG TCT TCA GGA TTC G-3′ |
| CD90 | Forward | 5′-CTG ACC CGT GAG ACA AAG AAG-3′ |
| | Reverse | 5′-TTG TAT TTG CTG AAG TTG G-3′ |
| CD117 | Forward | 5′-GAC AGG CTC TTC TCA ACC ATC T-3′ |
| | Reverse | 5′-AAG tCT GAT TTT CCT GGA TGG A-3′ |
| GAPDH | Forward | 5′-CGT CTT CAC CAC CAT GGA AGA-3′ |
| | Reverse | 5′-CGG CCA TCA CGC CAC AGT TT-3′ |

5. Immunoblotting

In order to measure expression levels of a protein of interest, equivalent amounts of proteins were separated by 10% sodium dodecylsulfate-polyacrylamide gel electrophoresis (SDS-PAGE) and transferred onto a polyvinylidene fluoride (PVDF) membrane at 25 V for 10 min with the aid of Semi-Dry transfer system (ATTO). Subsequently, the membrane was blocked for 2 hours with 5%(w/v) skimmed milk in Tris-buffered saline (pH 7.6) containing 0.1% Tween-20 (TBST), washed, and incubated overnight at 4° C. with a primary antibody in a blocking solution (normal horse serum, Vector laboratories). After being washed with TBST, the membrane was incubated with a suitable secondary antibody and washed again. A protein of interest was detected using EzWestLumi and luminol substrate (ATTO) on ImageQuant LAS-4000 (GE Healthcare, Uppsala, Sweden). The antibodies used in the analysis are summarized in Table 3:

TABLE 3
Antibody Used in Immunoassay

| | | | Application | | |
|---|---|---|---|---|---|
| Antigen (host) | Company | Cat. No | IF | ELISA | IB |
| Endiglin (mouse) | Santa Cruz | Sc-18838 | 1:200 | — | |
| Flag (rabbit) | Sigma- | F7425 | 1:400 | — | |
| | Aldrich | | | | |
| CD44 (rabbit) | Abcam | ab51037 | — | 1:200 | — |
| RAGE (goat) | Abcam | ab7784 | 1:400 | 1:1,000 | |
| APP (rabbit) | Millipore | 07-667 | 1:200 | | 1:200 |
| BACE1 (mouse) | Millipore | MAB5308 | 1:200- | — | 1:1000 |
| Iba1 (goat) | Abcam | ab5076 | 1:500 | — | |
| IL-1β (rabbit) | Santa Cruz | sc-7884 | — | — | 1:100 |
| NF-kB p65 (rabbit) | Abcam | an97726 | — | — | 1:500 |
| (phosphor 5529) | | | | | |
| iNOS (rabbit) | BD | 610332 | — | — | 1:100 |
| | Bioscience | | | | |
| Arg1 (rabbit) | Santa Cruz | sc-20150 | — | — | 1:200 |
| AGE (rabbit) | Abcam | ab23722 | | 1:1,000 | 1:100 |
| HMGB1 (rabbit) | Abcam | ab18256 | — | 1:1,000 | — |
| S100 β (rabbit) | Abcam | ab52642 | — | 1:500 | — |
| SAPK/JNK (rabbit) | Cell | 9252 | — | | 1:1000 |
| | signaling | | | | |
| pSAPK/JNK | Cell | 9251 | — | — | 1:1000 |
| (rabbit) | signaling | | | | |
| Caspase 3 (rabbit) | Cell | 96625 | — | — | 1:1000 |
| | signaling | | | | |
| Caspase 8 (rabbit) | Cell | 47905 | — | — | 1:1000 |
| | signaling | | | | |
| Caspase 9 (mouse) | Cell | 9508 | — | — | 1:1000 |
| | signaling | | | | |
| β-actin (rabbit) | Abcam | ab8227 | — | — | 1:4000 |
| GAPDH (mouse) | Millipore | MAB374 | — | — | 1:1000 |
| Peroxidase labeled | Vector | PI2000 | — | — | 1:5,000 |
| anti-mouse IgG | | | | | |
| Peroxidase labeled | Vector | PI1000 | — | 1:1,000 | 1:5,000 |
| anti-rabbitIgG | | | | | |
| Alexa Fluor 555 | Invitrogen | A31572 | 1:500 | — | — |
| donkey anti rabbit | | | | | |
| IgG | | | | | |
| Alexa Fluor 488 | Invitrogen | A11055 | 1:500 | — | — |
| donkey anti goat | Invitrogen | A11001 | 1:500 | — | — |
| IgG | | | | | |

IF: Immunofluorescence, IB: Immunoblotting

6. Immunofluorescence

Frozen brain tissue sections (10 μm) were immersed in 1% normal serum to block the binding of the antibodies to non-specific antigens and incubated overnight at 4° C. with the antibodies (see Table 3). The brain sections were incubated for 1 hour with a fluorolabeled secondary antibody and washed with PBS. The nuclei were counterstained with DAPI (4′6-diamino-2-phenylindole; Sigma-Aldrich) at mom temperature for 5 min. Fluorescent signals generated was detected on a confocal microscope (LSM 710, Carl Zeiss, Oberkochen, Germany). Analysis of detected fluorescent signals was conducted using Image J software (NIH, Bethesda, Md.).

7. ELISA (Enzyme-linked immunosorbent assay) and Sandwich ELISA

All ELISA reagents, working standards, and specimens used for measuring sRAGE secretion from MSCs and sRAGE-MSC were prepared according to the manufacturer's instruction (Aviscera Bioscience, Santa Clara, Calif.). The specimens were added to wells of antibody-precoated microplates. Subsequently, antibody conjugates listed in Table 3 were added to respective wells and incubated, followed by measuring optical densities at 450 nm on a microplate reader. In order to analyze the expression of RAGE ligands and interaction between RAGE and ligands thereof in vivo and in vitro, each well of 96-well microplates was coated overnight AGE, HMGB1, and S1000 antibodies in 100 mM carbonate/bicarbonate buffer (pH 9.6) at 4° C. The wells were washed with 0.1% triton x-100 in PBS (TPBS) and treated with 5% skim milk at room temperature for 2 hours to block protein binding sites. After a wash with PBS, other tissue extracts, cell lysates, or supernatant samples were added to the wells and incubated overnight at 4° C. Washing with TPBS was followed by incubation of the specimens with an RAGE antibody at room temperature for 2 hours in the wells. Again, the plates were washed with TPBS and the specimens were then incubated with peroxidase-conjugated secondary antibody at room temperature for 2 hours. Subsequently, TMB substrate solution was added and incubated for 5 to 10 min before the addition of an equivalent volume of stop solution (2N H₂SO₄). Optical densities were read at 450 nm. Concentrations of the antibodies used are listed in Table 3.

8. FACS (Fluorescence-Activated Cell Sorting)

The MSC markers CD44 (positive), CD73 (positive), and CD34 (positive) were examined using FACS to identify MSCs and sRAGE-MSCs. Cells were incubated with fluorescein isothiocyanate (FITC)-labeled primary antibody for 1 hour in a dark condition and then washed three times with PBS. After staining, 10⁶ MSCs or sRAGE-MSCs were subjected to FACS (Calibur, BD Bioscience) analysis.

9. Preparation of Experimental Animal

In the following Examples, 7-week-old Sprague Dawley (SD) rats were used. The animals were individually caged and allowed to freely access standard foods and water, with a 12/12 hrs light/dark cycle in a temperature-controlled (24° C.) facility. Animal experiments were conducted according to the international guidelines of the Institutional Animal Care and Use Committee (AAALAC International) at Gachon University.

10. Reagents

Human AP protein fragment 1-42 (Aβ₁₋₄₂; DAEFRHDSGY EVHHQKLVFF AEDVGSNKGA IIGLMVGGVV IA; SEQ ID NO: 5; Sigma-Aldrich; cat. A9810) was dissolved at a concentration of 4 mM in dimethylsulfoxide (DMSO). Human sRAGE protein (UniProtKB acc.no. Q15109) was purchased from Biovendor (cat. RD172116100, Brno, Czech Republic; SEQ ID NO: 6 (Total 339AA. MW: 36.5 kDa. UniProtKB accession no. Q15109. N-Terminal His-tag 14AA)) and dissolved at a concentration of 0.5 mg/ml in acetate buffer (pH 4). The prepared Aβ₁₋₄₂ peptide and sRAGE were stored at −80° C. until use. Just before use, the Aβ₁₋₄₂ peptide and the sRAGE were diluted to respective concentrations of 200 nM and 6.7 nM in PBS.

11. Alzheimer's Disease (AD) Animal Model

Before surgical operation, SD rats (Reference Example 9) were anesthetized with Zoletil 50 (50 mg/kg) and Rompun (10 mg/kg). The Aβ₁₋₄₂ peptide or the sRAGE was dissolved at 200 uM or 6.7 nM, respectively, in phosphate buffered saline (PBS). After dissection of the midline on the head skin, a hole was drilled at the position 8.3 mm posterior to and 5.4 lateral to the bregma of the skull, using a biological electric drill. Then, a 5 μl Hamilton syringe with a 30-guage needle was vertically put down to the target region (4.5 mm deep). Under stereotaxic guidance, reagents and cells were injected to ENT as follows: 5 μl of 200 μM Aβ₁₋₄₂ solution, 3 μl of 6.7 nM sRAGE protein, 5 μl of 10⁶ sRAGE-MSCs or 10⁶ MSCs.

Several minutes after injection of the Aβ₁₋₄₂ solution to the target region, the sRAGE protein, MSCs, or sRAGE-MSCs were injected. The reagents were slowly injected at a speed of 1 μl/min to prevent the countercurrent thereof. After completion of the injection, the needle was slowly withdrawn and the surgical region was closed with a wound clip.

Normal, healthy rats which had not been injected with the Aβ₁₋₄₂ were used as a control.

12. qRT-PCR (Quantitative Polymerase Chain Reaction)

Using PrimeScript 1^{st} strand cDNA Synthesis Kit (TAKARA), cDNA (complementary DNA) was synthesized from the isolated brain RNA. qRT-PCR was performed using CFX386 touch (Bio-rad, Hercules, Calif.), with the reaction efficiency and the number of threshold cycles determined with CFX386 software. All the primers used were designed on the basis of human specific sequences, which are as shown in Table 2, above.

13. TUNEL (TdT-Mediated dUTP-X Nick End Labeling)

TUNEL performed on frozen brain sections washed with PBS, using In Situ Cell Death Detection Kit (TUNEL; Roche Applied Science, Burgess Hill, UK). Briefly, tissue sections were incubated for 2 minutes on ice with permeabilization solution (0.1% (w/v) sodium citrate solution containing 0.1% (w/v) Triton X-100). After being rinsed with PBS, the tissue sections were incubated with a TUNEL reaction mixture for 2 hours in a humidified chamber atmosphere (37° C. and dark conditions). The sections were then washed three times with PBS, mounted on a glass slide with the aid of a Vectashield mounting medium (Vector Laboratories, Burlingame, Calif.), and covered with coverslip. Apoptotic cell ratios were determined using Image J (NIH) software.

14. Cresyl Violet Staining and Neuron Counting

The frozen rat brain tissue slides prepared previously were dried at room temperature for 5 min, washed with PBS for 10 min, incubated in graded ethanol series (100% (v/v) ethanol: 3 min, 90% ethanol 3 min, 80% ethanol: 3 mins, 70% ethanol 5 min), and then washed with distilled water. Thereafter, the tissues were stained for 20 minutes with 0.1% cresyl violet staining solution (Sigma-Aldr ich) containing glacial acetic acid, followed by washing with distilled water, 70% ethane for 1 min, 80% ethanol for 30 sec. 90% ethanol for 20 sec. 100% ethanol for 20 sec., and fmally with xylene for 5 min. On an optical microscope (Carl Zeiss), histological analysis (BBC biochemical, Mt Vernon, Wash.) was made of the sections that were mounted using an OpticMount mounting medium. Neurons were counted using Image J software (NIH).

15. Statistical Analysis

In consideration of the small sample size, a non-parametric analysis was used. Group comparisons were performed through the Mann-Whitney test using IBM SPSS statistics 23 software (SPSS, Inc., Armonk, N.Y.) (Null hypotheses of no difference were rejected when p-values were <0.05). Data were expressed as averages of the measurements in three independent tests.

### EXAMPLE 1

### Characterization of sRAGE-MSCs (Normal MSC Characteristics Present)

After testing for characterization, the sRAGE-secreting MSCs (sRAGE-MSC) established in the Reference were identified to have normal MSC characteristics.

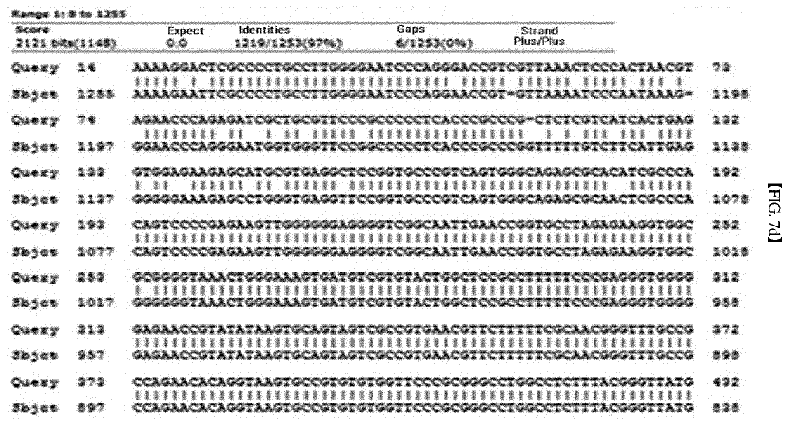

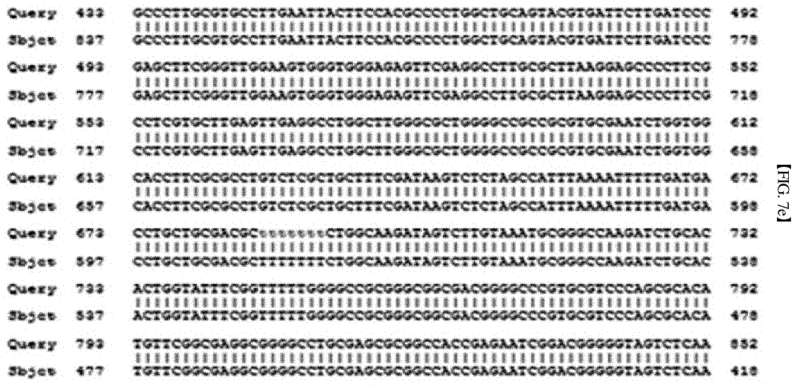

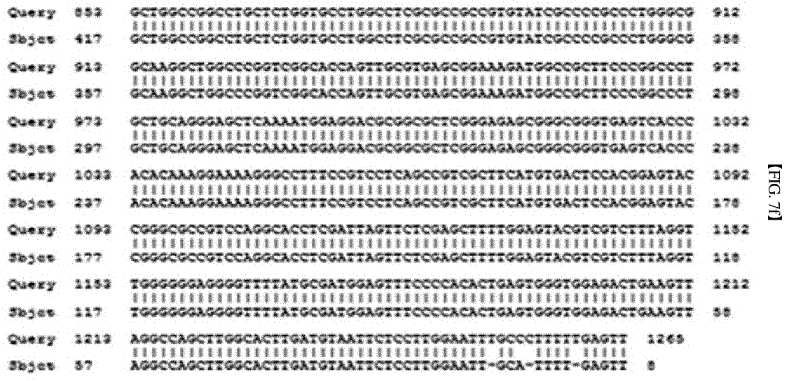

First of all, as explained supra, the CRISPR/Cas9 system was used to induce MSCs to secrete sRAGE and sRAGE was labeled with Flag (see FIG. 1a). Intrinsic and artificially synthesized sRAGE (FIGS. 7a-c and 7e-f) were evaluated for sequence homology by sequencing. The genomic DNA of sRAGE-MSC was examined by junction PCR and the results are shown in FIG. 1b. As shown in FIG. 1b, RAGE expression was confirmed.

Examination was made of the intracellular and extracellular sRAGE expression (presence) of sRAGE-MSCs by ELISA, immunoblotting, and immunofluorescence analysis, and the results are shown in FIGS. 1c to 1g.

FIG. 1c depicts results of ELISA analysis for quantitation of sRAGE secreted from sRAGE-MSCs and MSCs, showing that the amount of sRAGE secreted by sRAGE-MSCs is 892.80 times greater than that secreted by MSCs.

FIGS. 1d and 1e show immunoblotting results accounting for sRAGE-conjugated Flag expression levels in sRAGE-MSC supernatant (d) and cell lysate (e).

FIG. 1f provides dual staining confocal microscopic images showing expressions of the sRAGE conjugated Flag (Flag, red) and stem cell markers (Endoglin, green) (Scale bar=100 μm, magnification=200×; Nuclei were stained with DAPI and appear blue). As shown in FIG. 1f, MSCs and sRAGE-MSCs expressed the MSC marker Endoglin and sRAGE synthesized by sRAGE-MSCs was observed in the cytoplasm of MSCs and formed small red particles.

FIG. 1g is a graph showing Flag intensities in representative confocal microscopy images of FIG. 1f (**, <0.01 versus MSC, ***, <0.001 versus MSC). As shown in FIG. 1g, the Flag expression intensity of sRAGE-MSCs was 5.02 times higher than that in MSCs.

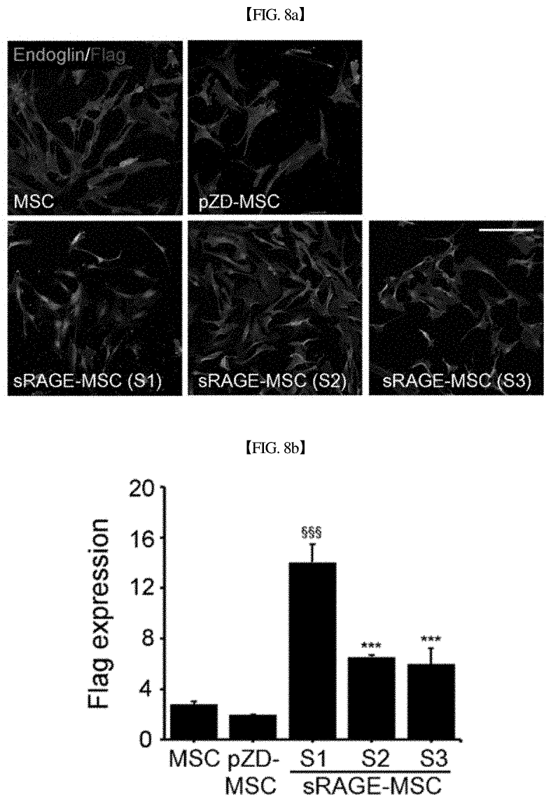

FIG. 8a shows confocal microscopic images of sRAGE-conjugated Flag (red), nuclei (DAPI, blue), and MSC-specific markers (Endoglin, green) in MSC, pZD-MSC, and subcultured sRAGE-MSCs (S1, S2, and S3) and FIG. 8b is a graph in which Flag expression is quantified from the representative result of FIG. 8a. During sRAGE-MSC proliferation in cell culture plates, as shown in FIGS. 8a and 8b, the Flag steadily decreased in local expression level, but remained high in intensity (FIGS. 8a and 8b).

FIG. 1h provides cytograms showing expression levels of positive stem cell markers (CD44, CD73) and negative markers (CD34) of sRAGE-MSCs. As shown in FIG. 1h, sRAGE-MSCs expressed characteristic MSC-specific markers despite the genetic modification thereof. The flow cytometry results of FIG. 1h indicate that positive markers including CD44 and CD73 are expressed in both sRAGE-MSCs and MSCs while the negative marker CD34 is not expressed in either the cell lines.

### EXAMPLE 2

### Assay 1 for Effect of sRAGE—sRAGE Increases Viability of Transplanted Cells by Downregulating RAGE Expression

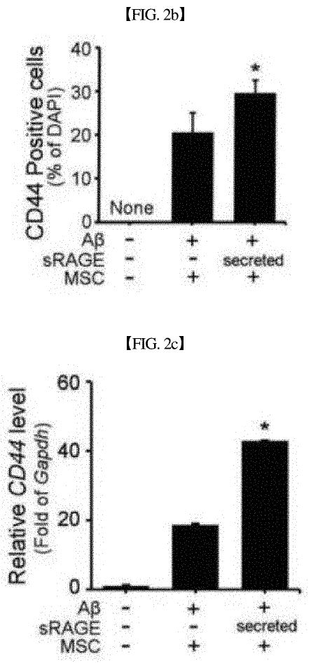

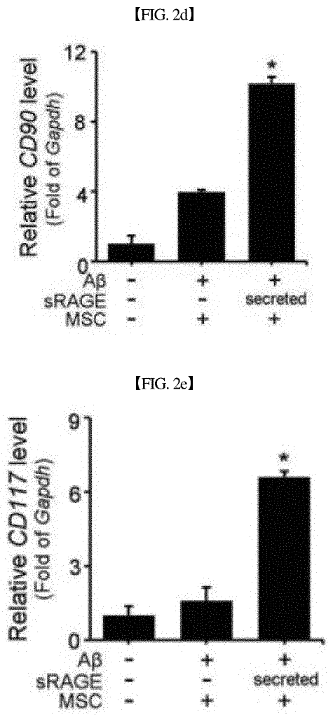

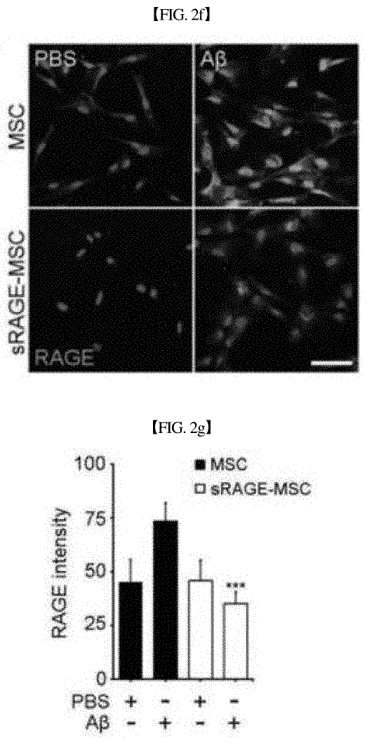

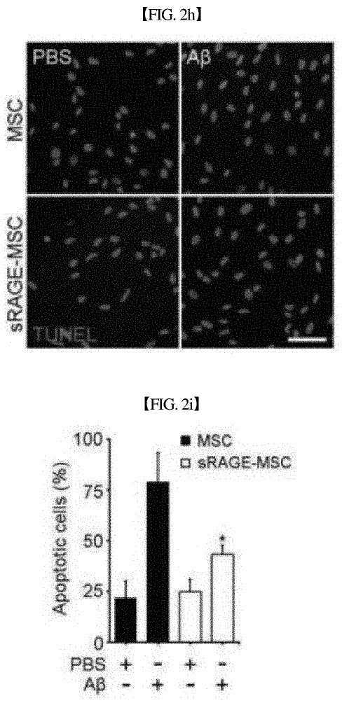

sRAGE-MSCs or MSCs were transplanted into the brains of Aβ₁₋₄₂-injected rats to assay the effect of sRAGE-MSCs Immunfluorescence and QRT-PCR were performed using human specific antibodies (see Table 3) and primers (see Table 2) to obtain fluorescence images four weeks after transplantation of sRAGE-MSCs and MSCs and to measure cell viability. The results are shown in FIGS. 2a to 2e.

FIG. 2a depicts immunofluorescence images accounting for the distribution of CD44-positive cells (red) as analyzed by immunofluorescence and FIG. 2b is a graph showing the number of CD44-positive cells. FIGS. 2c to 2e are graphs of expression levels of the human specific stem cell markers (CD44 gene (2c), CD90 gene (2d), and CD117 gene (2e)) as analyzed by qRT-PCR ((*, <0.05 versus MSC treated Aβ₁₋₄₂ injected rat brains). As shown in FIGS. 2a to 2e, the number of human specific CD44-expressing MSCs (CD44-positive cells) was 1.43 times higher for the sRAGE-MSC transplantation than for MSC transplantation (FIGS. 2a and b), and the expression levels of CD44, CD90, and CD117 mRNAs were also 2.31 times, 2.77 times, and 4.08 times higher in sRAGE-MSC, respectively (FIGS. 2c-2e). The results indicate that sRAGE-MSCs is higher in viability than MSCs at the same dose.

FIG. 2f depicts fluorescence images accounting for RAGE expression (green) in MSCs and sRAGE-MSCs after treatment with 1 μM Aβ₁₋₄₂ or PBS for 96 hrs., as analyzed by immunofluorescence and FIG. 2g is a graph in which the intensity of immunofluorescence obtained in FIG. 2f is quantitated. In order to compare viability between MSCs and sRAGE-MSCs, as shown in FIGS. 2f and 2g, RAGE expression in MSCs and sRAGE-MSC was confirmed and RAGE-related cell death was examined in vitro. After treatment of MSCs and sRAGE-MSCs with 1 μM Aβ₁₋₄₂ for 96 hrs, RAGE expression intensity increased by 64.97 folds for MSCs, compared to PBS, but by 25.78 folds for sRAGE-MSCs, with the increment of intensity 2.52 time lower than that for MSCs.

FIG. 2h depicts fluorescence images accounting for apoptosis (red) of MSCs and sRAGE-MSCs, as analyzed by TUNEL, and FIG. 2i is a graph showing the percentages of apoptotic cells to total cells. After Aβ₁₋₄₂ treatment in the same conditions, as shown in FIGS. 2h and 2i, the proportion of apoptotic cells increased up to 79.00% for MSCs, but was ceased at 43.19% for sRAGE-MSCs, which is 1.83 times lower than that for MSCs. These results show that RAGE blockade by secreted sRAGE increases the viability of sRAGE-MSC in the brains of Aβ₁₋₄₂-injected rats.

### EXAMPLE 3

### Assay 2 for Effect of sRAGE—sRAGE-MSCs Downregulate Expression of APP and BACE1 in Brains of Aβ₁₋₄₂-Injected Rats

Aβ₁₋₄₂ was injected into the ENT region of rats to create an Alzheimer's disease rat model. Aβ₁₋₄₂ injections were observed to increase the levels of amyloid precursor protein (APP) and beta-site APP cleaving enzyme 1 (BACE1).

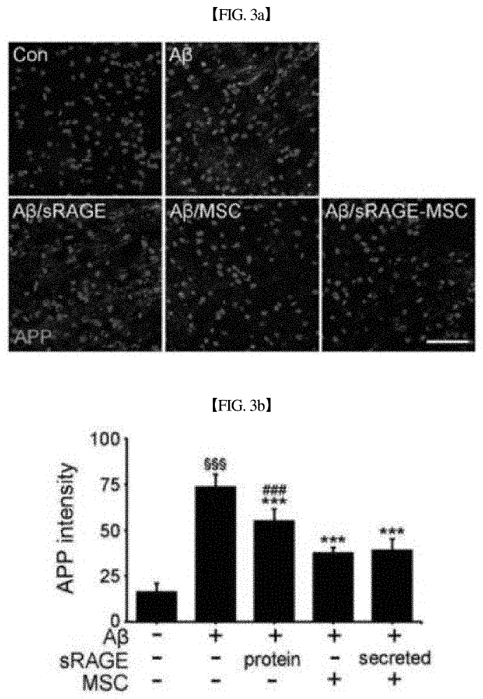

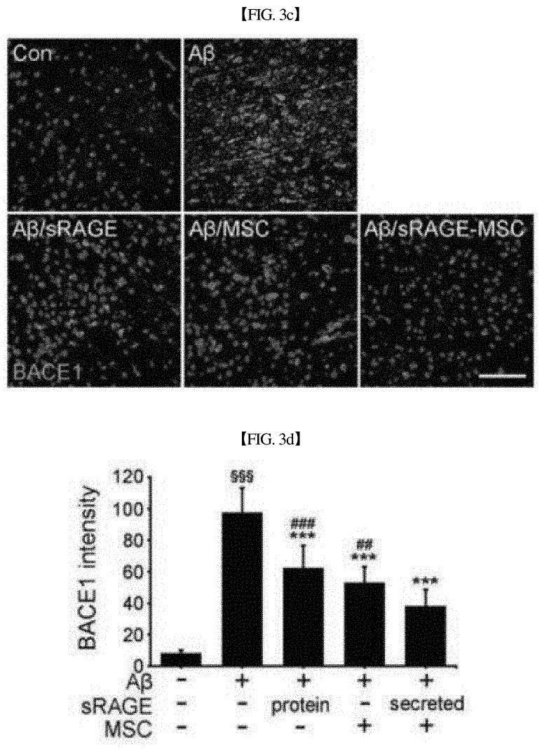

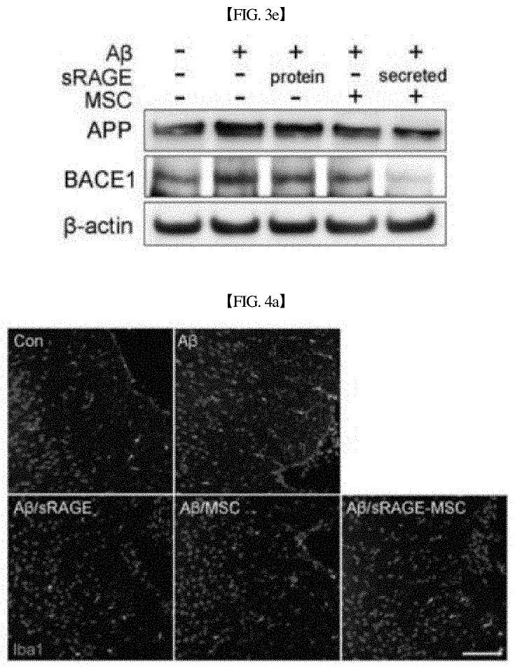

Expression levels of APP and BACE1 were measured by immunofluorescence, and the results are shown in FIGS. 3a to 3d.

FIG. 3a depicts fluorescence images accounting for APP expression (green), observed under a confocal microscope (Scale bar=100 μm), and FIG. 3b is a graph showing mean values after quantifying APP expression (green) fluorescence intensities observed in FIG. 3a for the cells. As shown in FIGS. 3a and 3b, post-Aβ₁₋₄₂ injection APP intensity (expression level) was 4.46-fold increased, compared to pre-Aβ₁₋₄₂ injection, whereas the intensity was 1.13-fold decreased for treatment with Aβ₁₋₄₂ and sRAGE, compared to Aβ₁₋₄₂ injection. In addition, the APP intensity was decreased by 1.96 folds for Aβ₁₋₄₂ and and MSC treatment and by 1.88 folds for Aβ₁₋₄₂ and sRAGE-MSC treatment, as compared with Aβ₁₋₄₂ injection.

FIG. 3c depicts fluorescence images accounting for BACE1 expression (green) observed under a confocal microscope (Scale bar=100 μm), and FIG. 3d is a graph showing mean values after quantifying APP expression (green) fluorescence intensities observed in FIG. 3c for the cells. As shown in FIGS. 3c and 3d, BACE1 expression changes were similar to APP expression results after sRAGE-MSC treatment. Post-Aβ₁₋₄₂ injection BACE1 intensity was 12.10-fold increased, compared to pre-Aβ₁₋₄₂ injection, whereas the intensity was 1.57-fold decreased for treatment with Aβ₁₋₄₂ and sRAGE, compared to Aβ₁₋₄₂ injection. In addition, the BACE1 intensity was decreased by 1.87 folds for Aβ₁₋₄₂ and MSC treatment and by 2.61 folds for Aβ₁₋₄₂ and sRAGE-MSC treatment, as compared with Aβ₁₋₄₂ injection. The effect of decreasing BACE1 protein levels was more effective upon treatment with sRAGE-MSCs than sRAGE protein or MSCs.

FIG. 3e depicts APP and BACE1 protein levels in the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment, as measured by immunoblotting analysis. As shown in FIG. 3e, a remarkable decrease in the expression of APP and BACE1 was detected for a combination of Aβ₁₋₄₂ injection and sRAGE-MSC treatment, compared to Aβ₁₋₄₂ injection alone. The expression changes observed in FIG. 3E were similar to the results measured above in the immunofluorescence method.

### EXAMPLE 4

### Assay 3 for Effect of sRAGE—sRAGE-MSCs Reduce Microglial Activation and Downregulate Expression of RAGE Ligands and Inflammatory Proteins

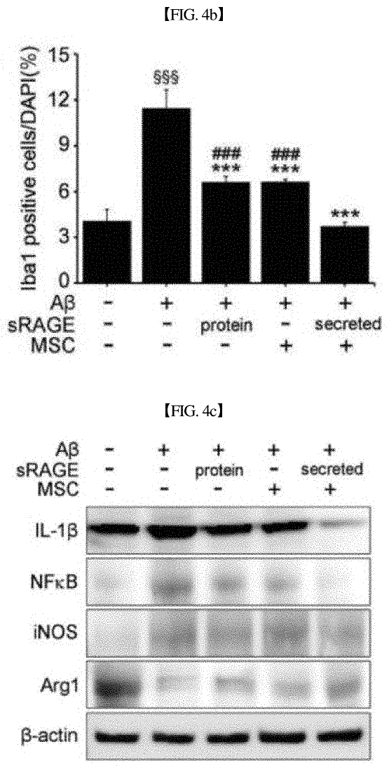

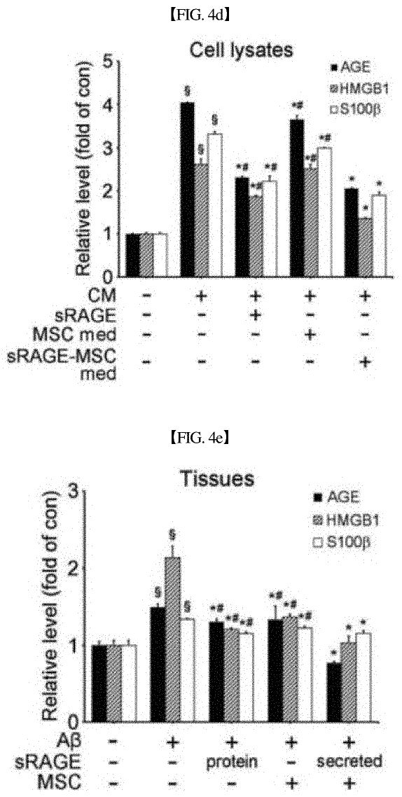

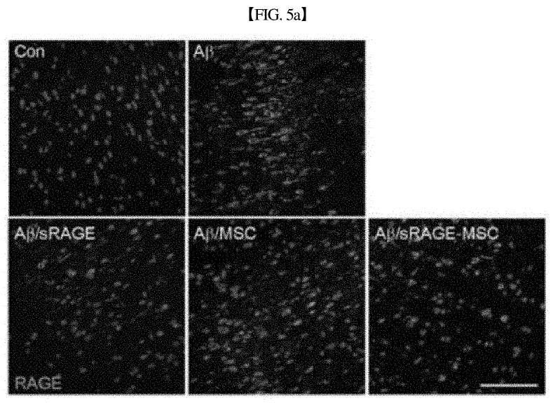

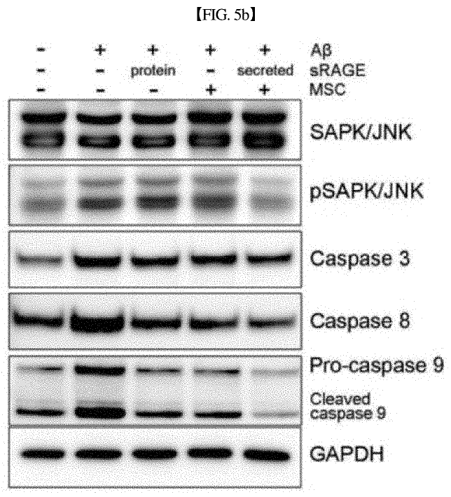

To investigate relationship between inflammatory proteins and activated microglial cells in the brains of Aβ₁₋₄₂ -injected rats, Iba1 (activated macrophage marker)-positive cells were counted to examine the distribution of activated microglia cells, and the results are shown in FIGS. 4a and 4b. FIG. 4a depicts confocal microscopic images accounting for the distribution of activated microglial cells (Iba1; green) in the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment and FIG. 4b is a graph showing the ratio of Ibal-positive cells to total cells expressed. As shown in FIGS. 4a and 4b, Ibal-positive cells in the brain of the Aβ₁₋₄₂-injected rats was 2.02 times more abundant than that of naive controls. A slightly decreased number of Ibal-positive cells was detected upon treatment with a combination of Aβ₁₋₄₂ and sRAGE protein or MSCs. However, treatment with a combination of Aβ₁₋₄₂ and sRAGE-MSCs significantly decreased the number of Ibal-positive cells, compared to Aβ₁₋₄₂ injection (3.08-fold lower).

Levels of inflammatory proteins such as IL-Iβ and NF-κB were measured and shown in FIG. 4c. FIG. 4c shows the expression levels of inflammatory proteins including IL-Iβ and NF-κB in the brains of Aβ₁₋₄₂-injected rats, as measured by immunoblotting analysis. As shown in FIG. 4c, levels of inflammatory proteins such as IL-Iβ and NF-κB were increased in the brains of Aβ₁₋₄₂-injected rats, but certainly decreased in the brains of rats having undergone a combination of Aβ₁₋₄₂ injection and sRAGE-MSC treatment, compared with Aβ₁₋₄₂ injection. Interestingly, sRAGE-MSCs can modulate M1 or M2 microglial cells in the brains of Aβ₁₋₄₂-injected rats. After treatment with Aβ₁₋₄₂ and sRAGE-MSC, the level of the M1 microglial marker iNOS decreased and the level of the M2 microglia marker Argl increased.

FIG. 4d is a graph showing levels of the RAGE ligands AGE, HMGB1 and S100β in HM06 cell lysates after treatment with CM, sRAGE protein, MSC medium (MSC med), or sRAGE-MSC medium (sRAGE-MSC med) for 24 hours, respectively, as analyzed by ELISA. FIG. 4e is a graph showing levels of AGE, HMGB1, and S100β in brain tissues of Aβ₁₋₄₂-injected rats. As shown in 4d and 4e, sRAGE-MSCs reduced expression levels of the RAGE ligands AGE, HMGB1, and S100β in activated microglial cells (as measured by ELISA in vivo and in vitro) as well as modulating inflammatory proteins and microglial cells. In in-vitro assays, RAGE ligands were synthesized from activated HMO6 induced by SH-SY5Y (neuronal cells) treated with 1 μM Aβ₁₋₄₂ for 96 hours. When HMO6 cells were co-treated with the CM (conditioned medium) of sRAGE-MSCs, the expression levels of the RAGE ligands were remarkably reduced, compared with treatment with sRAGE protein or MSC medium (FIG. 4d). Co-treatment with Aβ₁₋₄₂ and sRAGE-MSC more effectively decreased RAGE ligand levels in brain tissues than treatment with sRAGE protein or MSC (FIG. 4e). These results are similar to in vitro results.

### EXAMPLE 5

### Assay 4 for Effect of sRAGE-MSCs Protective Activity Against RAGE-Mediated Neuronal Apoptosis in Aβ₁₋₄₂-Injected Rat Brain

To assay the protective effect of sRAGE-MSCs on RAGE-mediated neuronal cell death, RAGE expression in the brains of Aβ₁₋₄₂-injected rats was examined by immunofluorescence. FIG. 5a depicts confocal microscopic images accounting for RAGE expression (green) in the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment. As shown in FIG. 5a, the fluorescence intensity indicating the expression of RAGE was increased by Aβ₁₋₄₂ injection, whereas co-treatment with Aβ₁₋₄₂ and sRAGE-MSC decreased the fluorescence intensity, compared with Aβ₁₋₄₂ injection. In addition, FIG. 5b shows SAPK/JNK, pSAPK/JNK, caspase 3, caspase 8, and caspase 9 in the brains of rat having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment, as measured by immunoblotting analysis. As shown in FIG. 5b, expression levels of RAGE-mediated neuronal apoptosis-related proteins such as SAPK/JNK. caspase 3, caspase 8 and caspase 9 were significantly lower in the brains of rats co-treated with Aβ₁₋₄₂ and sRAGE-MSCs, compared to the brains of Aβ₁₋₄₂ injected rats (FIG. 5b).

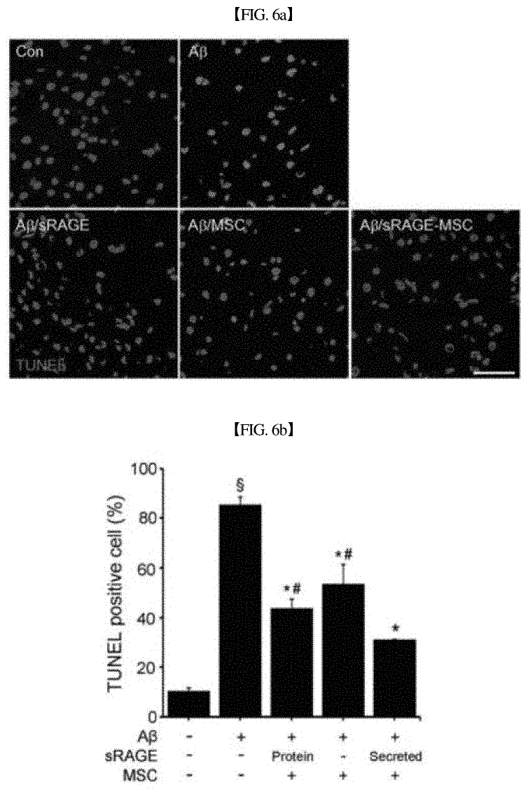

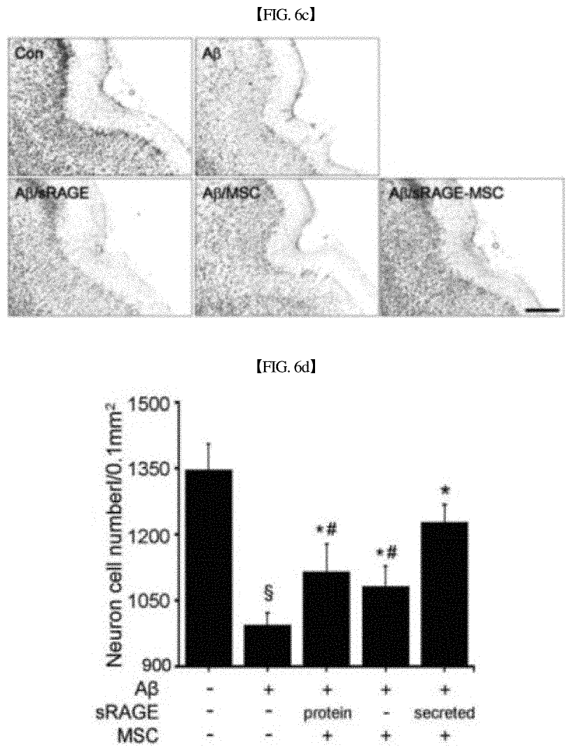

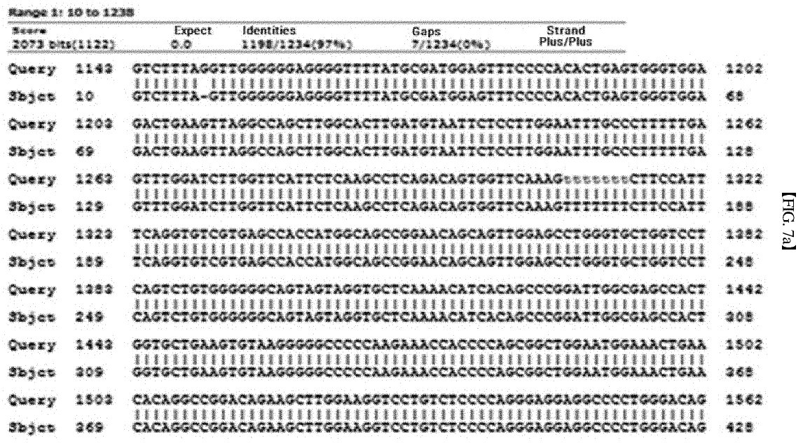

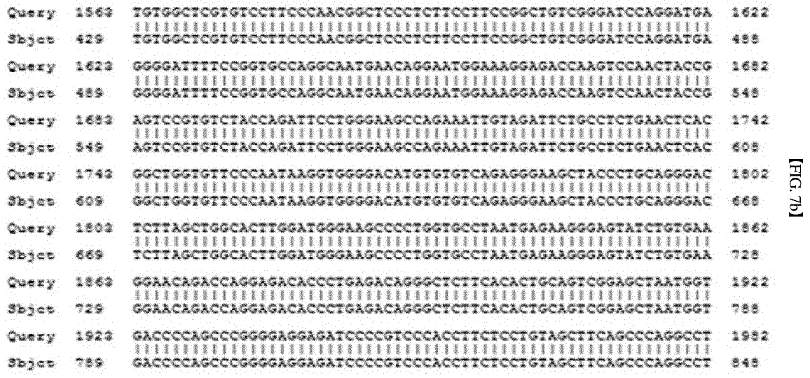

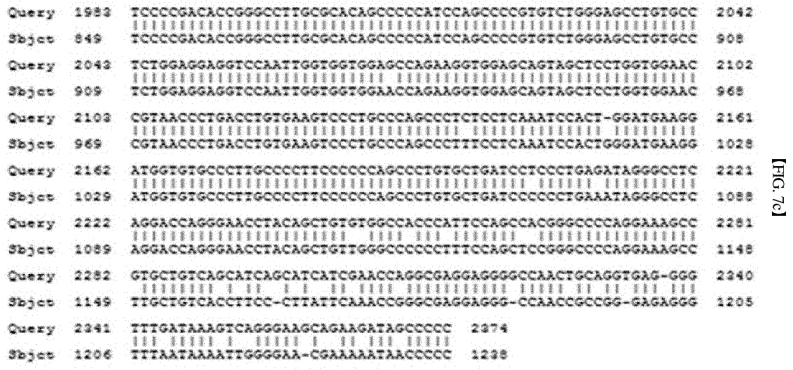

An assay was conducted to examine whether or not the increased expression levels of RAGE and RAGE-related cell death proteins in the brain of Aβ₁₋₄₂-injected rat is correlated with RAGE-mediated neuronal apoptosis and the results are shown in FIGS. 6a and 6b. FIG. 6a depicts confocal micrographic images of the brains of rats having undergone Aβ₁₋₄₂ injection, a combination of Aβ₁₋₄₂ injection and sRAGE protein treatment, a combination of Aβ₁₋₄₂ injection and MSC treatment, or a combination of Aβ₁₋₄₂ injection and sRAGE-MSC treatment and FIG. 6b is a graph showing counts of TNNEL-positive cells as measured by image J software. As shown in FIGS. 6a and 6b, it was confirmed by TUNEL analysis that the ratio (%) of TUNEL positive cells in the brains of Aβ₁₋₄₂ injected rats was higher than the Aβ₁₋₄₂-untreated control. The brains of the Aβ₁₋₄₂-injected rats had a significant decreased number of TUNEL positive cells when treated with sRAGE-MSCs, compared with sRAGE protein or MSC.

Finally, cresyl violet staining was performed to see if the sRAGE secreted by sRAGE-MSCs improves the viability of neurons in the brains of Aβ₁₋₄₂-injected rats. Images obtained from the cresyl violet staining are shown in FIG. 6c, and the quantification results thereof are given in FIG. 6d. As shown in FIGS. 6c and 6d, the brains of Aβ₁₋₄₂-injected rats showed lower numbers of live neurons than the control (Aβ₁₋₄₂ untreated group) whereas a significantly increased number of neurons was detected in the brains of Aβ₁₋₄₂-injected rats post treatment with sRAGE protein, MSCs, or sRAGE-MSC, compared to pre-treatment therewith. In addition, the sRAGE-MSC treatment of the number of living neurons in the brains of Aβ₁₋₄₂-injected rats was increased by 1.55 and 1.15 folds upon treatment with sRAGE-MSCs, compared to sRAGE protein and MSCs, respectively.

### EXAMPLE 6

### Preparation and Characterization of sRAGE-iPSC

In order to generate sRAGE-secreting iPSCs, sRAGE donor vector constructed by cloning a human EF1-α promoter, an sRAGE-encoding sequence, and poly A tail into the pZDonor vector (Sigma-Aldrich) (see FIG. 1a) was transfected, together with the CRISPR/CAS9 RNP, into iPSCs. The guide RNA was designed to target a safe harbor site known as AAVS1 on chromosome 19 (Cas9: derived from Streptococcus pyogenes (SEQ ID NO: 4), target site of sgRNA: gtcaccaatcctgtccctag (SEQ ID NO: 7)). Transfection was performed using a 4D nucleofector system (Lonza). Transfection conditions were as set forth in the Lonza protocol (cell type ‘hES/H9’) on the website. Electroporation was performed using P3 primary cell 4D nucleofector X kit L (Lonza, V4XP-3024). sRAGE-secreting iPSCs were created by transfecting 15 pg of Cas9 protein, 20 μg of gRNA, and 1 μg of the sRAGE donor vector into 2×10⁵ human iPSCs (Korean National Stem Cell Bank).

Three days after transfection, genomic DNA was isolated from the transfected iPSCs to determine the knock-in (KI) of sRAGE in the genomic DNA of the iPSCs. PCR primers were prepared with MVS1 Fwd (iPSC itself sequence) and Puro rev (insertion sequence) (AAVS1 FWD primer: CGG AAC TCT GCC CTC TAA CG; Puro Rev primer: TGA GGA AGA GTT CTT GCA GCT).

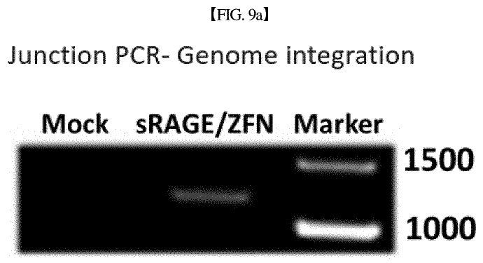

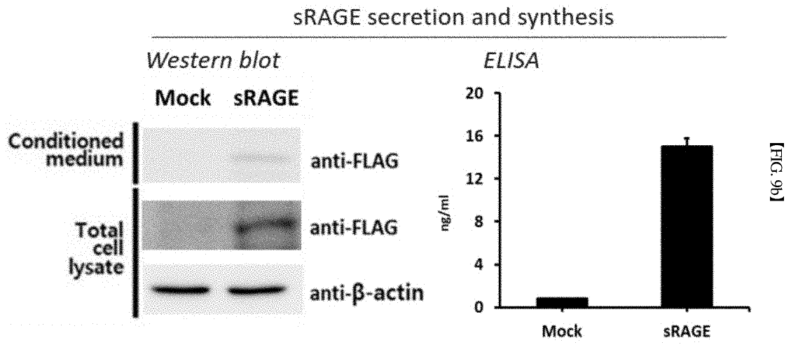

PCR is performed at 56° C. with 30 cycles. Following electrophoresis, the bands were observed under UV light. The obtained result is shown in FIG. 9a. FIG. 9a shows that the gene of sRAGE was successfully integrated at the MVS1 site.

Expression and secretion levels of sRAGE were confirmed by immunoblotting and ELISA.

First, immunoblotting was performed as follows: a whole cell lysate was prepared in RIPA (radio immunoprecipitation assay) lysis buffer (ATTA, WSE7420) and protease inhibitor cocktail (ATTA, WSE7420), followed by sonication. The prepared cell lysate was centrifuged at 17,000×g for 20 minutes at 4° C., and the supernatant was collected. Equal amounts (30 30 μg) of proteins were separated on 10% polyacrylamide gel and the separated proteins were transferred onto a nitrocellulose membrane (Millipore) at 200 mA for 2 hours. The membrane was treated with 5% non-fat skim milk for 1 hour at room temperature to block nonspecific antibody binding. The membrane was incubated overnight at 4° C. with a primary protein-specific antibody (Sigma, F-7425) and b-actin (Abeam, ab8227) and then with a secondary antibody at room temperature for 1 hour. After several washes, proteins were detected using enhanced chemiluminescence (ECL).

ELISA was performed as follows: the total soluble RAGE secreted was quantified using a human soluble receptor advanced glycation end products (sRAGE) ELISA kit (Aviscera Bioscience, S1(00112-02). Samples and 100 μl of the standard solution were added (in the reverse order of serial dilutions) to 96-well microplates pre-coated with a human sRAGE antibody and containing 100 μl of a diluted complete solution per well. The plates were then covered with a seal and incubated for 2 hours on a micro plate shaker at room temperature. After incubation, the solution was completely aspirated and washed four times with a wash solution. A dilution of a detection antibody in a working solution was added in an amount of 100 μl to each well after which the plate was covered with a seal and incubated on a microplate shaker at room temperature. Aspiration and washing was repeated again. A horse radish peroxidase (HRP)-conjugated secondary antibody was added in an amount of 100 μl to each well, followed by incubation for 1 hour on a microplate shaker at room temperature under a light-tight condition. Aspiration and washing was repeated again. Finally, 100 μl of the substrate solution is added to each well and reacted for 5-8 minutes before 100 μl of the stop solution was added to terminate the reaction. Optical density was measured using a micro plate reader set at 450 nm.

The results obtained by performing western blot and ELISA are shown in FIG. 9b. As can be seen from the western blot results of FIG. 9b, the expression of Flag was observed in pzDonor vector-transfected sRAGE-iPSCs. As understood from the ELISA result of FIG. 9b, which accounts for the total expression level of sRAGE secreted to the medium, sRAGE was detected at a concentration of 15.6 ng/ml in the culture medium of sRAGE-iPSCs, which was markedly high, compared to the level of sRAGE detected in the medium of mock-iPSC, 0.8 ng/ml.

1. A stem cell, which comprises a gene coding for soluble receptor for advanced glycation end products (sRAGE), and secrets sRAGE.

2. The stem cell of claim 1, wherein the stem cell are at least one selected from the group consisting of embryonic stem cells, adult stem cells, induced pluripotent stem cells (iPS cells), and progenitor cells.

3. The stem cell of claim 1, wherein the stem cell is an induced pluripotent stem cell or mesenchymal stem cell.

4-9. (canceled)

10. A method for prevention or treatment of Alzheimer's disease, the method comprising a step of administering the stem cell of claim 1 to a patient in need of preventing or treating Alzheimer's disease.

11. A method of inhibiting an expression of amyloid precursor protein (APP) or beta-site APP cleaving enzyme 1 (BACE1) in a patient with Alzheimer's disease, the method comprising a step of administering the stem cell of claim 1 to the patient.

12. A method of inhibiting an expression of a RAGE ligand or an inflammatory protein in a patient with Alzheimer's disease, the method comprising a step of administering the sRAGE-secreting stem cell of claim 1 to the patient.

13. A method of inhibiting RAGE-mediated neuronal death or inflammation in a patient with Alzheimer's disease, the method comprising a step of administering the sRAGE-secreting stem cells of claim 1 to the patient.

14. The method of claim 10, wherein the prevention or treatment of Alzheimer's disease is by at least one of the following activities:
inhibition of the expression of amyloid precursor protein (APP) or beta-site APP cleaving enzyme 1 (BACE1),
inhibition of the expression of a RAGE ligand or an inflammatory protein, and
inhibition of RAGE-mediated neuronal apoptosis or inflammation.

15. The method of claim 12, wherein the RAGE ligand is at least one selected from the group consisting of AGE (Advanced Glycation End products), HMGB1 (High mobility group box 1), and S100β.

16. A pharmaceutical composition comprising the stem cell of claim 1.


SEQUENCE LISTING (7 sequences)

SEQ ID NO: 1 - RNA, 12 nt, Artificial Sequence
  Essential part of crRNA
guuuuagagcua

SEQ ID NO: 2 - RNA, 10 nt, Artificial Sequence
  3′-terminal part of crRNA
ugcuguuuug

SEQ ID NO: 3 - RNA, 60 nt, Artificial Sequence
  Essential part of tracrRNA
uagcaaguuaaaauaaggcuaguccguuaucaacuugaaaaaguggcaccgagucggugc

SEQ ID NO: 4 - protein, 1368 aa, Artificial Sequence [sequence omitted]

SEQ ID NO: 5 - protein, 42 aa, Artificial Sequence [sequence omitted]

SEQ ID NO: 6 - protein, 339 aa, Artificial Sequence
  Human sRAGE protein, wherein the region of first 14 amino acids is N-Terminal His-tag
MRGSHHHHHHGMASAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGEGFDKVREAEDSPQHM

SEQ ID NO: 7 - DNA, 20 bp, Artificial Sequence
  Target site of human AAVS1
gtcaccaatcctgtccctag